(19) United States
(12) Patent Application Publication
(10) Pub. No.: US 2017/0238996 A1
(43) Pub. Date: Aug. 24, 2017

(54) MEDICAL TRACKING SENSOR ASSEMBLY

(71) Applicant: General Electric Company, Schenectady, NY (US)

(72) Inventors: Dan Stephen Frame, Salt Lake City, UT (US); Daniel Eduardo Groszmann, Belmont, MA (US); Laurent Jacques Node-Langlois, Salt Lake City, UT (US); Tobias Schroeder, Melrose, MA (US)

(21) Appl. No.: 15/052,763

(22) Filed: Feb. 24, 2016

(51) Int. Cl. A61B 34/20

(52) U.S. Cl. CPC A61B 34/20; A61B 2034/2051

(57) ABSTRACT

Methods and systems are provided for an electromagnetic coil assembly for a surgical navigation tracking system which may be used during image-guided surgery. A medical instrument assembly for an electromagnetic surgical navigation system, may comprise a tracking sensor interface included within a handle of a medical instrument, the interface having a first mating surface. Further, the assembly may include a tracking sensor including a second mating surface and one or more electromagnetic coils, where the tracking sensor is removably coupled to the tracking sensor interface of the medical instrument via an attachment device included on one or more of the first mating surface and second mating surface.

### FIELD

Embodiments of the subject matter disclosed herein relate to an electromagnetic tracking system, and more particularly, to an electromagnetic tracking system for use in image-guided surgery.

### BACKGROUND

Electromagnetic tracking systems have been used in various industries such as aviation, motion sensing, retail, and medicine to provide position and orientation information for objects. They employ electromagnetic coils as electromagnetic transmitters and receivers. The electromagnetic field generated by the transmitter may be sensed by the receiver and used to estimate a position and/or orientation of the receiver relative to the transmitter.

In medical applications, electromagnetic tracking systems have proven particularly useful because they can track medical instruments such as catheters and needle tips within a patient's body, without line-of-sight requirements. Thus, when a medical instrument is obscured from view, such as when it is inserted into a patient's body, its position and/or orientation can still be obtained and visualized via the electromagnetic tracking system. An operator (e.g., a physician, surgeon, or other medical practitioner) may therefore more precisely and rapidly adjust the position of the medical instrument within the patient's body during image-guided surgery.

The medical instruments used during image-guided surgery may be equipped with a first electromagnetic coil assembly including one or more first coils, while a patient reference assembly may include one or more second electromagnetic coils (and thus may be referred to herein as a second electromagnetic coil assembly). In some examples, the patient reference assembly may be coupled to the patient anatomy to serve as a reference point for the electromagnetic tracking system. An electrical current may be supplied to either the first or second coil assemblies, generating an electromagnetic field. The electromagnetic field may in turn cause changes in the outputs from the two coil assemblies due to the mutual inductance between the coil assemblies. The position and/or orientation of the medical instrument may then be estimated based on changes in the outputs of the two coil assemblies. Together, the two coil assemblies may therefore provide an image of the instrument location relative to the patient anatomy to the operator.

However, in examples where the first coil assembly is coupled to the medical instrument, it may take a significant amount of time to identify and calibrate the receiver for the specific instrument being used. Additionally, depending on the type of instrument being used, the design of the first coil assembly may need to be modified for proper attachment thereto. Such constrains require multiple different coil assembly designs which increases manufacturing costs. In other examples, where the coil assembly is included within the medical instrument, the cost of the medical instrument is increased, and the lifespan of coil assembly is reduced (e.g., may be limited to a one-time use).

### BRIEF DESCRIPTION

In one embodiment, a medical instrument assembly for an electromagnetic surgical navigation system, comprises a tracking sensor interface included within a handle of a medical instrument, the interface having a first mating surface, a tracking sensor including one or more electromagnetic coils, and a second mating surface, and an attachment device, adjustable to physically couple and decouple the tracking sensor and medical instrument via the first and second mating surfaces. In this way, the receiver may more easily be attached, detached, and reattached to different types of medical instruments. Further, a similar receiver design may be used in multiple types of medical instruments, thereby reducing the cost and complexity of such electromagnetic tracking systems.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example image-guided surgery system that may include an electromagnetic tracking system according to an embodiment of the invention.

FIG. 2A shows a schematic of a first example of an electromagnetic tracking system that may be included in the image-guided surgery system of FIG. 1 according to an embodiment of the invention.

FIG. 2B shows a schematic of a second example of the electromagnetic tracking system of FIG. 2A according to an embodiment of the invention.

FIG. 3 shows a flow chart of an example method for tracking a medical instrument during an image-guided surgery using an electromagnetic tracking system according to an embodiment of the invention.

FIG. 4 shows a flow chart of an example method for identifying a type of medical instrument used image-guided surgery according to an embodiment of the invention.

FIG. 5 shows an isometric side view of a first embodiment of a tracking sensor included within a medical instrument assembly according to an embodiment of the invention.

FIG. 6 shows a cross-sectional view of the first embodiment of the tracking sensor shown in FIG. 5, according to an embodiment of the invention.

FIG. 7 shows a top view of a first embodiment of a medical instrument included within a medical instrument assembly according to an embodiment of the invention.

FIG. 8 shows a cross-sectional view of a first embodiment of a medical instrument assembly including the tracking sensor shown in FIGS. 5-6, and the medical instrument shown in FIG. 7, according to an embodiment of the invention.

FIG. 9 shows an isometric side view of the first embodiment of the medical instrument assembly shown in FIG. 8, according to an embodiment of the invention.

FIG. 10 shows an isometric side view of a second embodiment of the tracking sensor according to an embodiment of the invention.

FIG. 11 shows a cross-sectional view of a second embodiment of the medical instrument assembly including the second embodiment of the tracking sensor shown in FIG. 10, according to an embodiment of the invention.

FIG. 12 shows an isometric side view of the second embodiment of the medical instrument assembly shown in FIG. 12, according to an embodiment of the invention.

FIG. 13 shows an isometric side view of a third embodiment of the tracking sensor according to an embodiment of the invention.

FIG. 14 shows an isometric side view of a third embodiment of the medical instrument assembly including the third embodiment of the tracking sensor shown in FIG. 13, according to an embodiment of the invention.

FIG. 15 shows a cross-sectional view of the third embodiment of the medical instrument assembly show in FIG. 14, according to an embodiment of the invention.

FIG. 16 shows an isometric side view of a fourth embodiment of the tracking sensor according to an embodiment of the invention.

FIG. 17 shows a cross-sectional view of a fourth embodiment of the medical instrument assembly including the fourth embodiment of the tracking sensor shown in FIG. 16, according to an embodiment of the invention.

FIG. 18 shows an isometric side view of an operator's hand holding a medical instrument assembly, such as any of the embodiments of the medical instrument assembly shown in FIGS. 8-9, 11-12, 14-15, and 17, in a first gripping position, according to an embodiment of the invention.

FIG. 19 shows an isometric side view of an operator's hand holding a medical instrument assembly, such as any the embodiments of the medical instrument assembly shown in FIGS. 8-9, 11-12, 14-15, and 17, in a second gripping position, according to an embodiment of the invention.

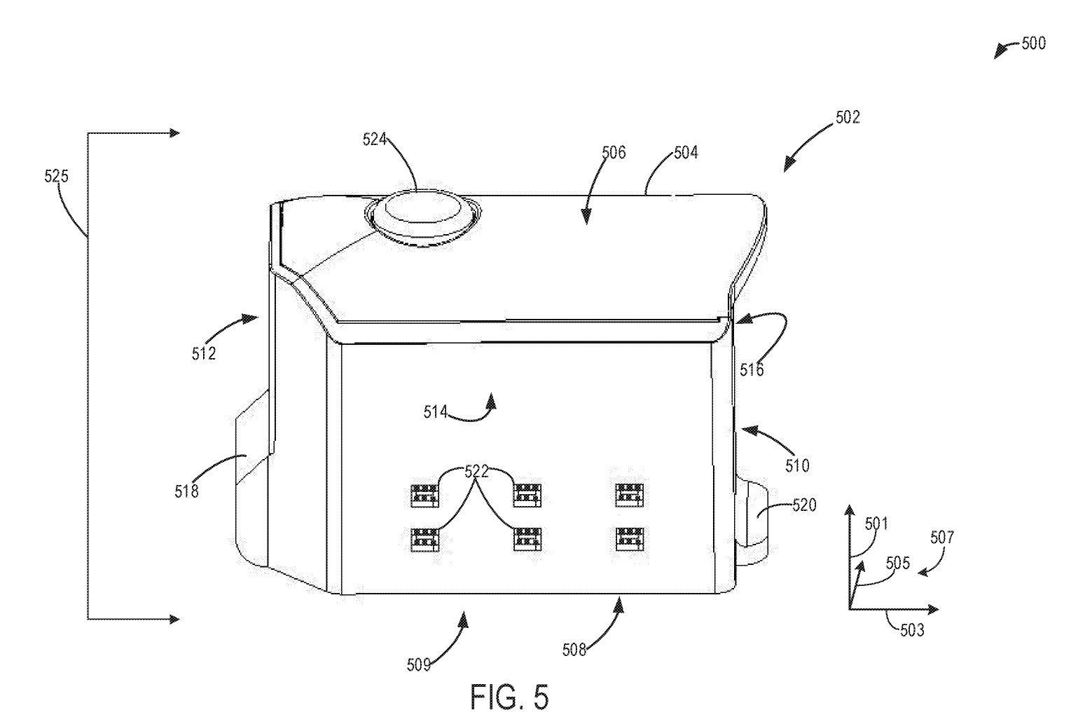

FIGS. 5-19 are drawn approximately to scale.

### DETAILED DESCRIPTION

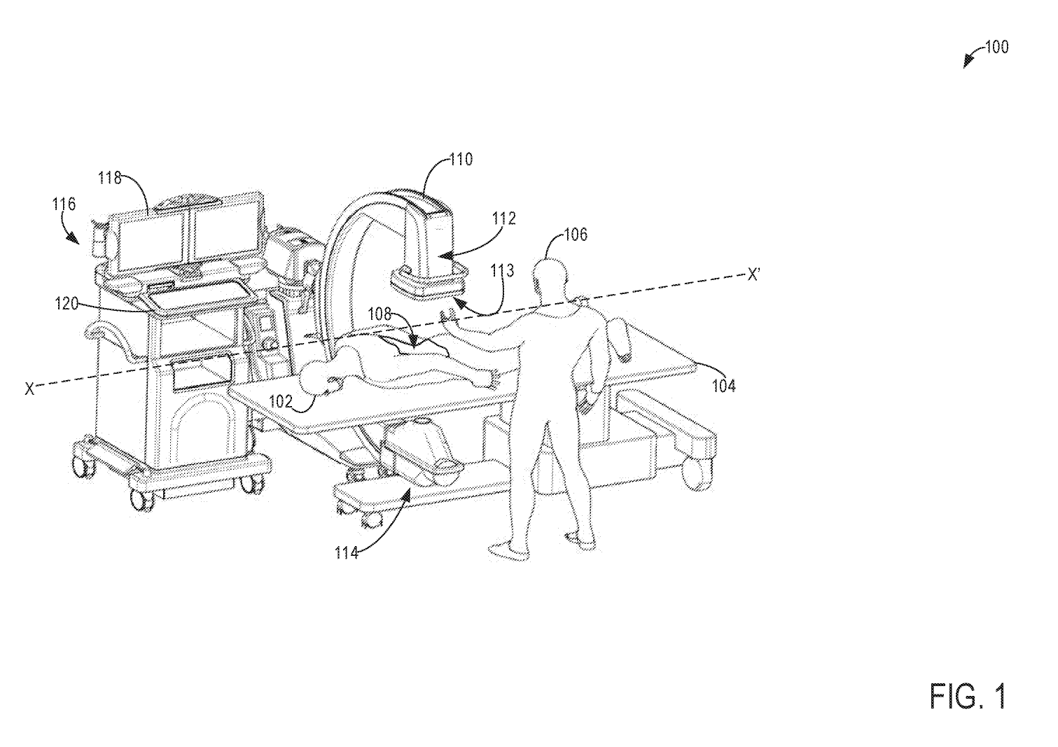

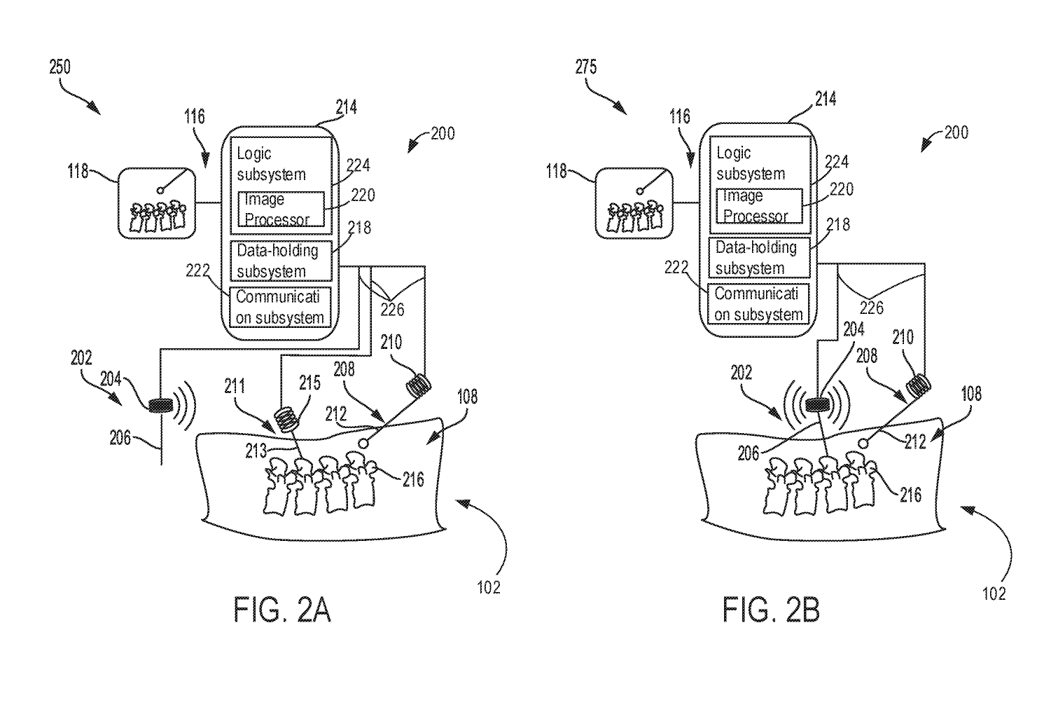

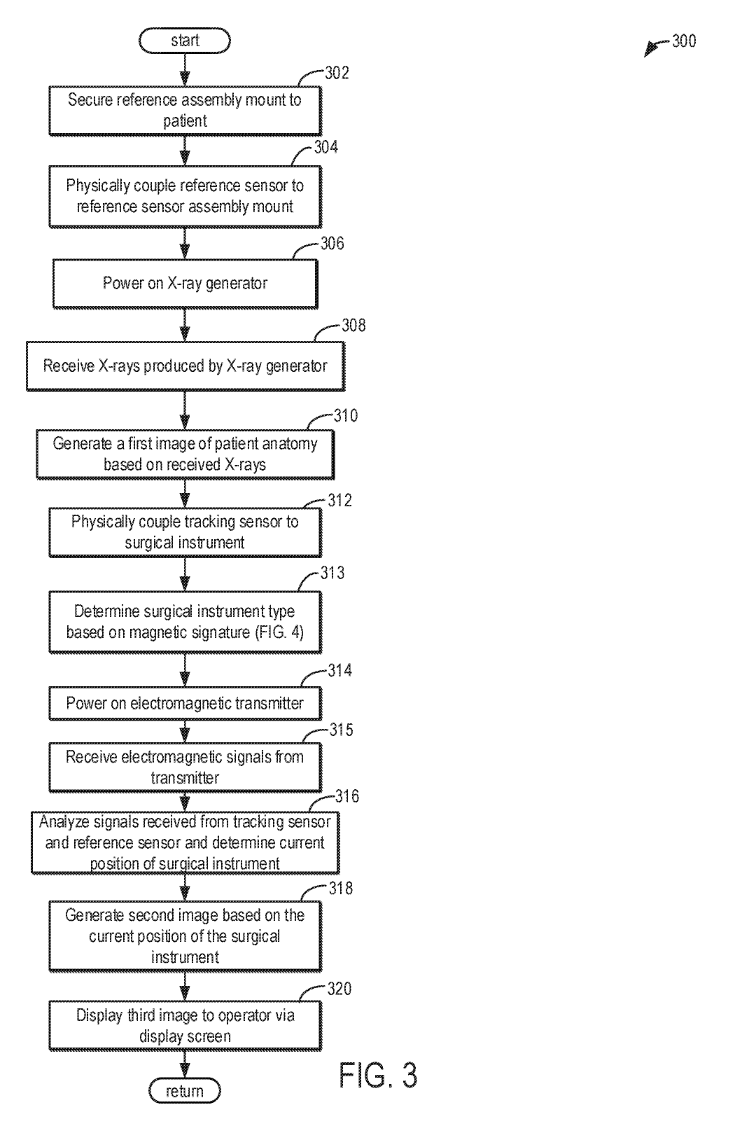

The following description relates to various embodiments of an electromagnetic tracking system for use in image-guided surgery. In some image-guided surgery systems, such as the example system shown in FIG. 1, a C-arm may be used to generate an X-ray image of a patient's anatomy. The electromagnetic tracking system, an example of which is shown in FIG. 2, may provide an indication of the current location of one or more medical instruments relative to the patient's anatomy. Specifically, estimations of the instruments' current positions may be overlaid onto the X-ray image of the patient's anatomy and displayed to an operator (e.g., surgeon, physician, medical practitioner) as described in the example method shown in FIG. 3. In this way, the operator may continue to adjust the position of the medical instruments within the patient's anatomy based on the displayed images of the instruments, even when the instruments are obscured from sight.

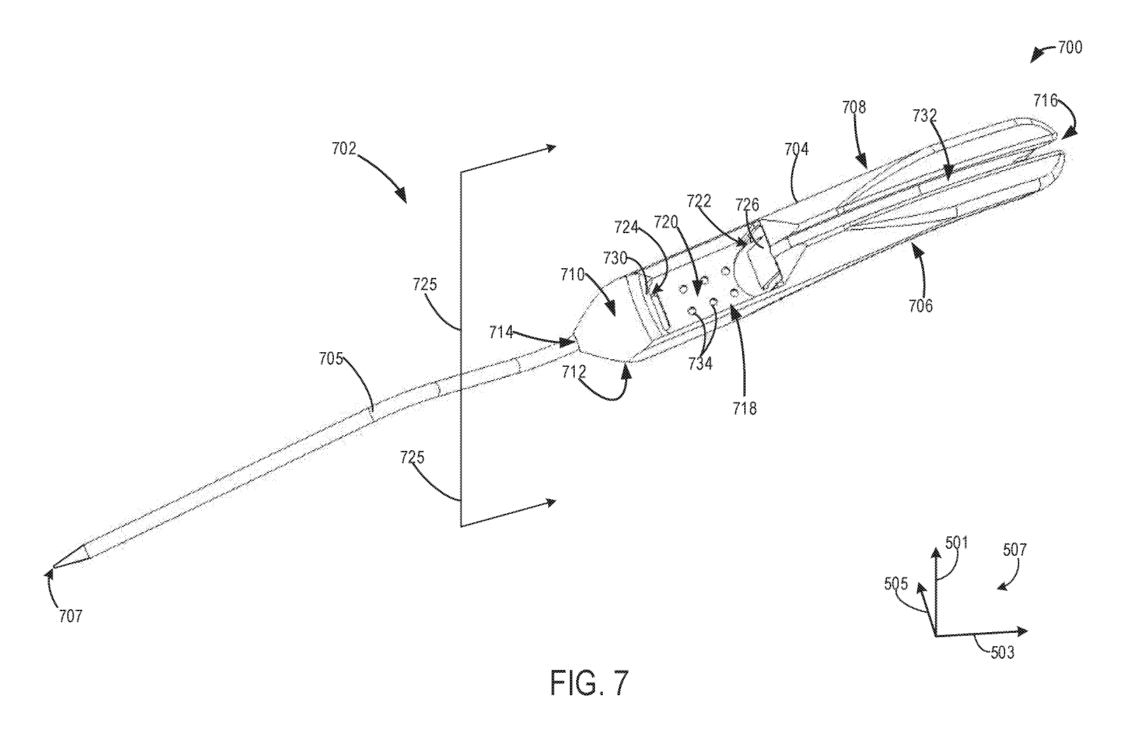

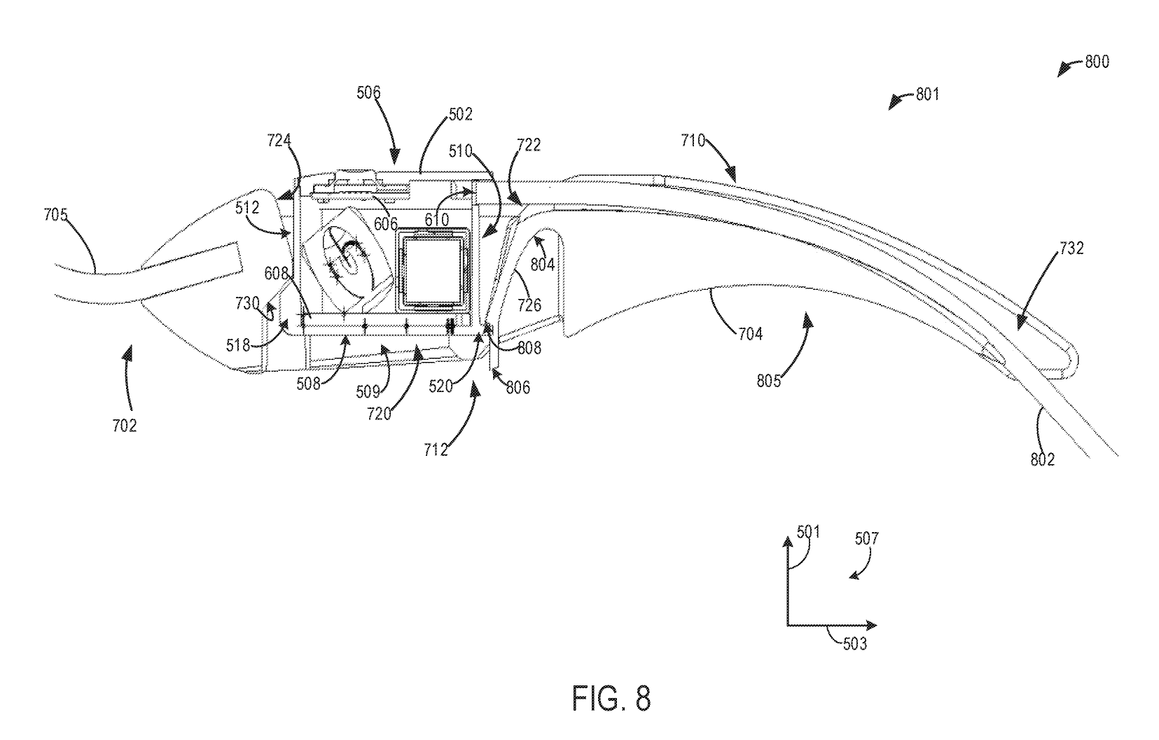

Specifically, the position of the medical instruments may be determined based on signals received from an electromagnetic transmitter and receiver, one of which may be removably coupled to the medical instrument. A first coil assembly may be included in a tracking sensor and may be coupled to the medical instrument to form a medical instrument assembly, such as the example assemblies shown in FIGS. 8-9, 11-12, 14-15, and 17. Specifically, the tracking sensor may be removably coupled to a handle of the medical instrument such as the handle shown in FIG. 7 via an attachment device.

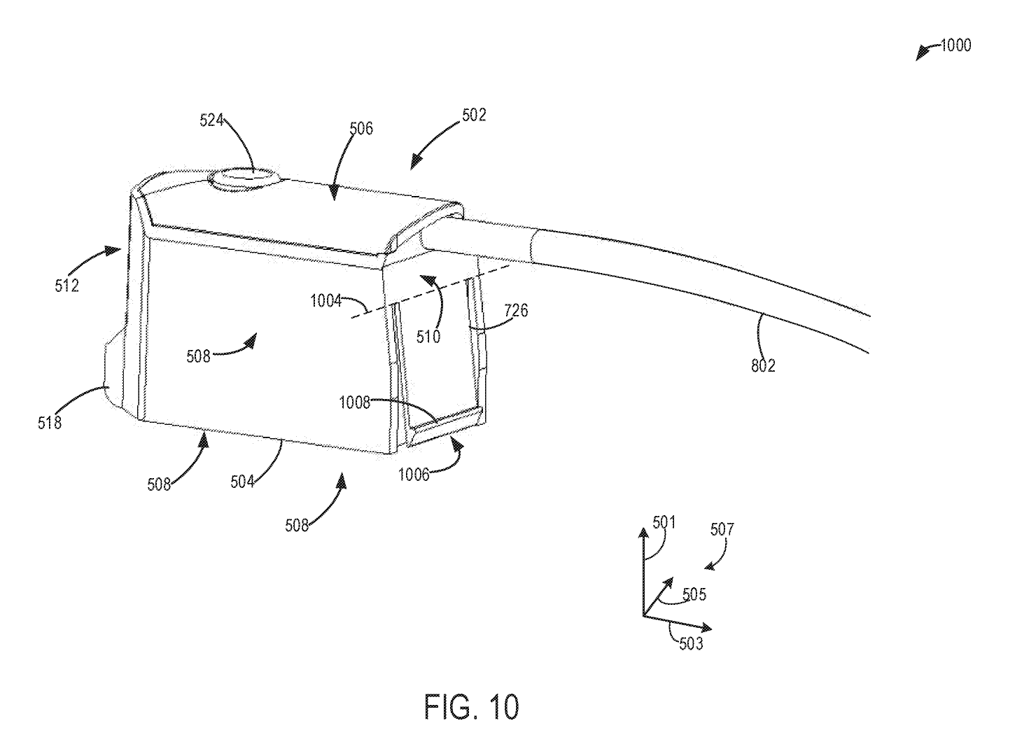

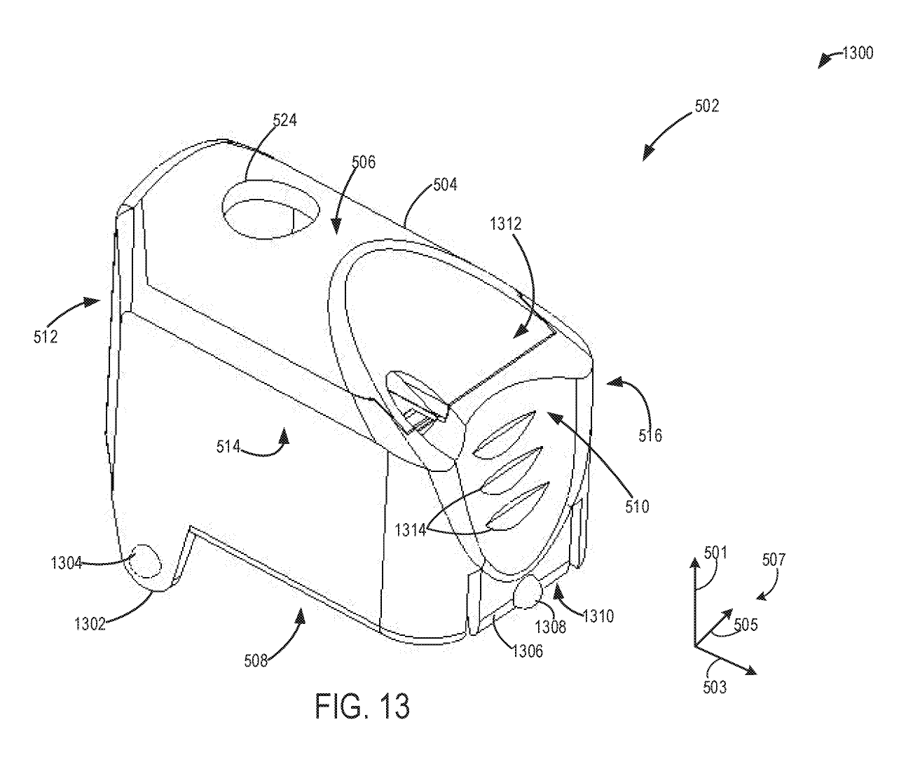

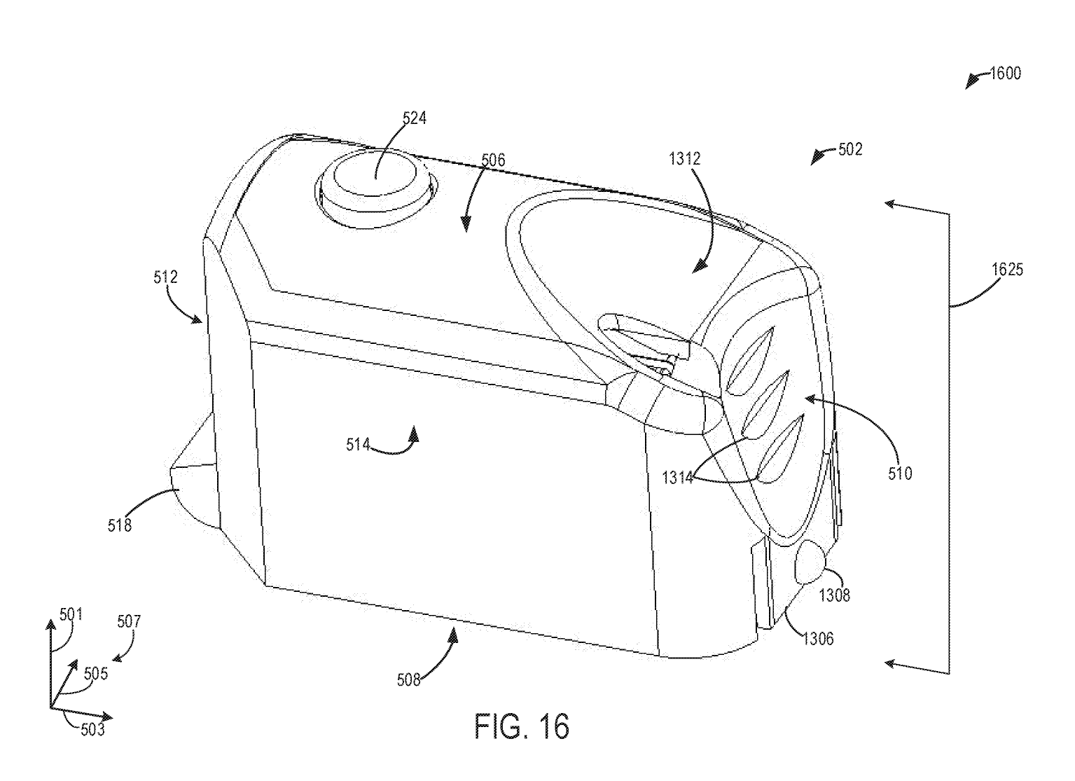

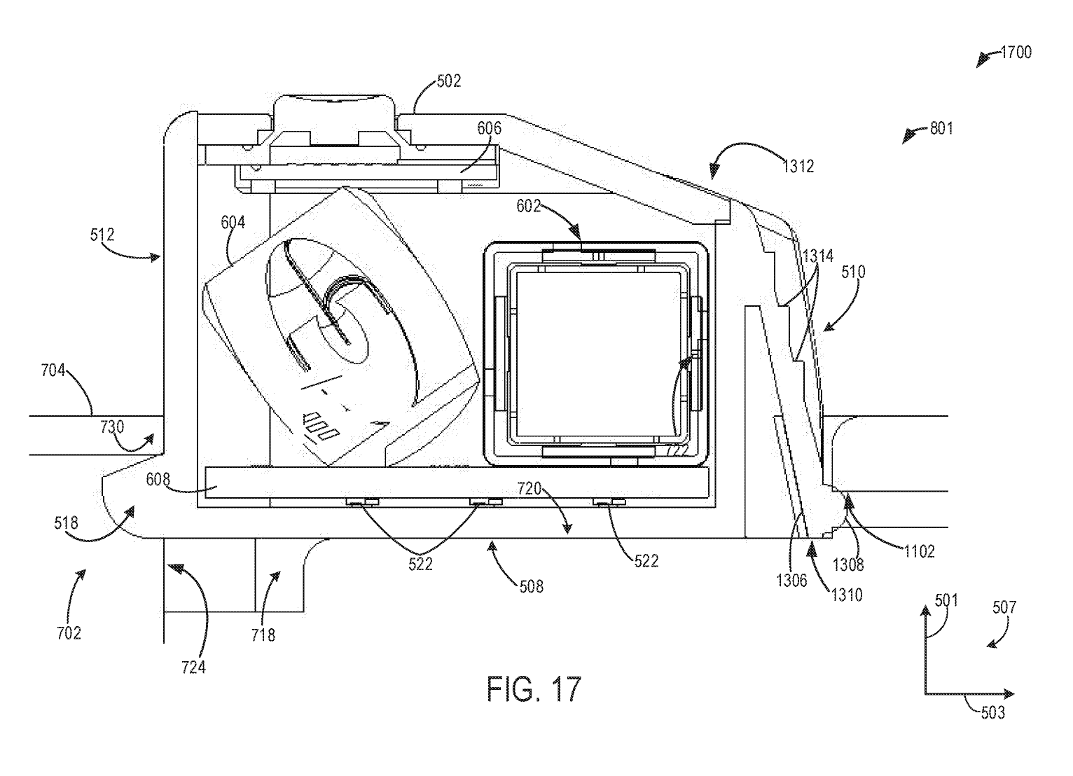

In some examples, such as those shown in FIGS. 5-9 the attachment device may be included on the handle of the medical instrument. However, FIGS. 10-17, depict alternate examples where the attachment device may be included on the receiver or portions of the attachment device may be included on both the receiver and handle. Specifically, FIGS. 10-12 show an example embodiment of the medical instrument assembly, where the attachment device is configured as a pivotable tab that extends from an end of the tracking sensor. In other embodiments, such as those shown in FIGS. 13-15, the tracking sensor may include two overhangs that permit rotation of the tracking sensor relative to the handle of the medical instrument. Further, FIGS. 16-17 show example embodiments of the medical instrument assembly where the tracking sensor includes a heel and pivotable tab positioned at opposite ends of the tracking sensor.

Although images of the patient anatomy may be obtained using a C-arm, it should be appreciated that the present techniques may also be useful when applied to images acquired using other imaging modalities, such as tomosynthesis, MRT, CT, and so forth. The present discussion of a C-arm imaging modality is provided merely as an example of one suitable imaging modality.

Beginning with FIG. 1, it shows a schematic of an example image-guided surgery system 100. The image guided surgery system 100 includes an electromagnetic tracking system (e.g., such as electromagnetic tracking system 200 described below with reference to FIG. 2) for aiding an operator 106 in performing surgery on a patient 102. The operator 106 may include one or more of a surgeon, physician, surgeon assistant, anesthesiologist, nurse, etc. Patient 102 may lie on table 104 positioned between an X-ray generator 114 and image intensifier or detector 112 of a C-arm 110. The C-arm 110 therefore comprises the generator 114 and detector 112, and generates an image of anatomy 108 of patient 102 based on outputs from the detector 112. The anatomy 108 may include a portion of the patient 102 which is one or more of undergoing surgery, exposed, to be operated on, etc., and as such anatomy 108 may also be referred to in the description herein as operating area 108. Patient 102 may be positioned on table 104, such that anatomy 108 is in-between the generator 114 and detector 112. In this way, when generator 114 is powered on, X-rays pass through anatomy 108 of patient 102.

The C-arm 110 may rotate about axis X-X′ when X-ray generator 114 is energized and emitting X-rays, to generate images of the patient anatomy 108 from multiple angles. Axis X-X′ may be approximately parallel to table 104. Thus, the C-arm 110 rotates around the table 104 and patient 102. An image of the patient anatomy 108 may be obtained based on outputs from the detector 112 during a portion or all of the rotational movement of the C-arm 110 while the generator 114 is powered on. That is, the C-arm may be rotated a threshold number of degrees to obtain an image of the patient anatomy 108 as described in greater detail below with reference to FIG. 3. Specifically, the C-arm 110 may generate a plurality of outputs during the rotating of the C-arm 110, and a three-dimensional image of the patient anatomy 108 may be obtained by compiling the plurality of outputs into a single image. In some examples, the C-arm 110 may be rotated approximately 180 degrees when obtaining an image of the patient anatomy 108. However, in other examples the C-arm 110 may be rotated more or less than 180 degrees. In yet further examples, the C-arm may not be rotated and may remain approximately stationary when obtaining an image of the patient anatomy 108. In such examples where the C-arm 110 remains substantially stationary when the generator 114 is powered on, a single projection image (e.g., two-dimensional image) of the patient anatomy 108 may be obtained.

The X-ray generator 114 produces X-ray radiation that may penetrate the patient anatomy 108 and pass on to the detector 112. As the X-rays pass through the patient 102, the intensity of the X-rays may be attenuated to different degrees. These differences in X-ray intensity may be detected and/or amplified by the detector 112 across a surface 113 of the detector 112. Thus, an image of the patient anatomy 108 may be generated based on the relative intensities of received X-rays distributed across the surface 113 of the detector 112. A computing system 116 may receive and/or process the image data corresponding to the patient anatomy 108 from the detector 112, and may display an image of the anatomy 108 on a display screen 118.

In some examples, the detector 112 may be an analog image intensifier that converts the X-rays received from generator 114 into visible light. In such examples, the surface 113 of the detector 112 may comprise a fluorescent surface which illuminates in response to excitation by X-rays. The brightness or intensity of the surface 113 depends on the intensity of the X-rays striking the surface 113. Thus, as the X-rays generated by the generator 114 strike the surface 113 of the detector 112, the surface 113 may glow or illuminate in proportion to the intensity of the X-rays. Further, the detector 112 may include a camera positioned behind the surface 113. The camera captures a picture of the visible light produced by surface 113 in response to X-ray excitation, and this image is then sent to an image processor and/or storage component of computing system 116.

However, in other examples, the detector 112 may be configured as a flat-panel detector that converts the intensity of received X-rays directly into a digital value. In such examples, the detector 112 does not include a camera, and an image of the patient anatomy 108 may be generated based on the digital signals output from the detector 112. The digital signals output by the detector 112 therefore correspond to the relative intensities of the detected X-rays distributed across surface 113. From the detector 112, the digital signals may be sent to an image processor and/or storage component of the computing system 116. Based on the digital signals received from the detector 112, the computing system 116 may generate an image of the patient anatomy 108 and display the image on display screen 118.

The display screen 118 may be any suitable display screen such as cathode ray tube (CRT), LED, LCD, plasma display, etc. The display screen 118 may positioned such that it faces the operator 106. In this way, the operator 106 can identify and check anatomical details on the images displayed by the display screen 118, such as blood vessels, bones, kidney stones, the position of implants and instruments, etc. Further, the operator 106 may monitor instrument position by watching the display screen 118 during image-guided surgery.

The computing system 116 may include the image processor and various other components such as random access memory (RAM), keep alive memory (KAP), processors, logic subsystems, data-holding subsystems, servers, software instructions, etc., as described in more detail below with reference to FIG. 2. Further, the computing system 116 may include one or more user input devices 120 such as keyboards, mice, buttons, touch screen displays, etc. As described in greater detail below with reference to FIGS. 2 and 3, the computing system 116 may construct a three-dimensional image of the patient anatomy 108 based on outputs from the C-arm 110 obtained during rotation of the C-arm 110, when the generator 114 is powered on. Alternatively, the computing system 116 may construct single projection images of the patient anatomy 108 in examples where the C-arm remains substantially stationary with respect to the patient anatomy 108 when the generator 114 is powered on. Additionally, the computing system 116 may determine a current position and/or orientation of one or more medical instruments based on outputs from an electromagnetic tracking sensor coupled to the medical instrument. The computing system 116 may overlay an image of the current positions of the one or more medical instruments onto the X-ray image or three-dimensional tomographic image of the patient anatomy 108. This composite image of the patient anatomy 108 and medical instrument position may then be displayed on the display screen 118 to the operator 106.

Turning now to FIGS. 2A and 2B, they show schematics of examples of an electromagnetic tracking system 200 that may be used in an image guided surgery system, such as the image guided surgery system 100 described above with reference to FIG. 1. As such, components of the image guided surgery system 100 described above in FIG. 1 and numbered similarly in FIGS. 2A and 2B may be not be reintroduced or described again in the description of FIGS. 2A and 2B herein. FIGS. 2A and 2B depict example surgical conditions of patient anatomy 108, where tracking system 200 is included to monitor the positions of one or more medical instruments. As depicted in FIGS. 2A and 2B bone 216 of the anatomy 108 is exposed and may be used to physically secure components of the electromagnetic tracking system 200.

The electromagnetic tracking system 200 includes an electromagnetic transmitter and one or more electromagnetic receivers. Specifically, the transmitter may generate a magnetic field when current is provided. The magnetic field produced by the transmitter may induce current to flow in the one or more receivers. Based on the mutual inductances of the transmitter and receiver, a position of the one or more receivers relative to the transmitter may be estimated. Electromagnetic coils that may serve as either the transmitter or the receiver may be included within and/or coupled to each of a medical instrument and a patient reference sensor. Outputs from the electromagnetic coils (e.g., changes in current and/or voltage within the coils) may therefore provide an indication of the position and/or orientation of the instrument relative to the patient anatomy.

FIG. 2A shows a first schematic 250 of an example of the tracking system 200 where an electromagnetic patient reference assembly 202 is positioned proximate and external to the patient anatomy 108. In such examples, a reference coil assembly or reference receiver sensor 211 may be included in the tracking system 200 to increase the accuracy of the system 200. However, FIG. 2B shows a second schematic 275, depicting an alternate example of the tracking system 200, where the patient reference assembly 202 is positioned within and/or coupled to the patient anatomy 108. In such examples, the additional reference receiver sensor may not be included in the tracking system 200. Thus, in some examples, where the patient reference assembly 202 is coupled to the patient anatomy 108, the patient reference assembly 202 may serve as the reference sensor.

The patient reference assembly 202 may comprise a patient reference sensor 204 and a mount (also referred to herein as a mounting platform) 206 that physically secures the patient reference assembly 202 (e.g., to the anatomy 108 in the example shown in FIG. 2B). Thus, the mount 206 may secure the patient reference assembly 202 so that the patient reference assembly 202 is substantially stationary. Said another way, the mount 206 may physically couple the patient reference assembly to restrict and/or prevent movement of the patient reference assembly 202.

In the example shown in FIG. 2A, the mount 206 may be physically coupled to the patient 102 at a location external to the patient anatomy 108. However, in other examples, the mount 206 may be physically coupled to a location external to the patient 102. For example, the mount 206 may not be coupled to the patient 102 and may instead be coupled to a stationary object external to the patient 102 (e.g., bedside table).

Mount 206 may comprise an attachment interface for securing the mount to the external location such as a pin, clamp, screw, adhesive plate, etc. The patient reference sensor 204 may in some examples be selectively coupled to, and decoupled from, the mount 206. That is, the patient reference sensor 204 may be removably coupled to the mount 206. However, in other examples, the patient reference sensor 204 may be permanently secured to the mount 206. In such examples, the patient reference sensor 204 and mount 206 may be integrally formed as a single component.

Similarly, a medical instrument assembly (also referred to herein as instrument tracking assembly) 208 may include a medical instrument 212 and a first coil assembly or tracking sensor 210. The tracking sensor 210 may in some examples be selectively coupled and decoupled from the medical instrument 212 as described below in FIGS. 5-17. That is, the tracking sensor 210 may be removably coupled to the medical instrument 212. Specifically, the tracking sensor 210 may be removably coupled to a handle of the medical instrument 212. The medical instrument 212 may include one or more of forceps, clamps, retractors, distractors, scalpels, lancets, dilators, suction tips and tubes, injection needles, drills, endoscopes, tactile probes, screw inserters, awls, taps, rod inserters, pedicle probes, etc. However, in other examples, the tracking sensor 210 may be permanently secured to the medical instrument 212. In such examples, the tracking sensor 210 and instrument 212 may be integrally formed as a single component.

Further, the receiver sensor 211 may include an electromagnetic receiver 215 and a mount 213. The receiver 215 may in some examples be selectively coupled to and decoupled from the mount 213. That is, the receiver 215 may be removably coupled to the mount 213. However, in other examples, the receiver 215 may be permanently secured to the mount 213. In such examples, the receiver 215 and mount 213 may in some examples be integrally formed as a single component. The receiver 215 may in some examples be the same and/or similar to the tracking sensor 210. However, in other examples, the receiver 215 may be different than the tracking sensor 210. In yet further examples, the receiver 215 may be the same and/or similar to the reference sensor 204.

The reference receiver sensor 211 may be physically coupled to the patient anatomy 108 via the mount 213. Mount 213 may comprise one or more of a pin, clamp, screw, adhesive plate, etc. In some examples, the mount 213 may be physically secured to bone 216 of the patient anatomy 108. However, in other examples, the mount 213 and reference receiver sensor 211 may be physically secured to another portion of the anatomy 108 such as skin, organs, muscle, fat, etc.

Patient reference sensor 204 may comprise any suitable electromagnetic coil arrangement for generating and/or sensing an electromagnetic field. In some examples, the patient reference sensor 204 may be configured as an electromagnetic transmitter. Thus, the patient reference sensor 204 may comprise a second coil assembly, where coils included in the patient reference sensor 204 may generate electromagnetic waves when current flows there-through. In some examples, current may be provided by the computing system 116 via one or more electrical cables. However, in other examples, the patient reference sensor 204 may receive electrical power from another source such as a wall socket, battery, generator, etc. In still further examples, the patient reference sensor 204 may include its own power source, such as a battery. The current may be one or more of DC or AC current. In some examples, the patient reference sensor 204 may be directly electrically coupled to the computing system 116 via one or more electrical cables 226, as shown in FIGS. 2A and 2B.

However, in other examples, the patient reference sensor 204 may be wirelessly connected to the computing system 116 (e.g., via Bluetooth, Wifi, etc.).

One or more of the electrical power, current, and voltage supplied to the patient reference sensor 204 may be adjusted to regulate one or more of the intensity, frequency, and wavelength of electromagnetic waves generated by the patient reference sensor 204. In a preferred embodiment the electromagnetic waves generated by the patient reference sensor 204 may be radio waves. However, the frequency of the electromagnetic waves may be altered as desired by adjusting the current supplied to the patient reference sensor 204. Further, the computing system 116 may monitor one or more of the current, voltage, and power of the transmitter via 204, via the direct electrical connection.

The electromagnetic waves generated by the patient reference sensor 204 may be detected by one or more of the tracking sensor 210 of tracking assembly 208 and the receiver 215 of reference receiver sensor 211. Specifically, the electromagnetic field generated by the patient reference sensor 204 may induce current to flow in the tracking sensor 210 and/or receiver 215. The induced current flow in one or more of the tracking sensor 210 and/or receiver 215 may in turn generate electromagnetic fields that induce a change in the current flow in the patient reference sensor 204 (mutual inductance). Thus, the mutual inductances of one or more of the tracking sensor 210, receiver 215, and patient reference sensor 204 may cause changes in current flow therein, and therefore changes in outputs from one or more of the tracking sensor 210, receiver 215, and patient reference sensor 204.

The induced electrical outputs of one or more of the tracking sensor 210 and, receiver 215, and reference sensor 204 may then be used to determine a position of the tracking sensor 210 relative to the patient reference sensor 204. In some examples, outputs from both the patient reference sensor 204 and tracking sensor 210 may be used to determine a position and/or orientation of the tracking sensor 210 relative to the patient reference sensor 204. However in yet further examples, outputs from all of the patient reference sensor 204, receiver 215, and tracking sensor 210 may be used to determine a position and/or orientation of the tracking sensor 210 relative to the patient reference sensor 204. The receiver 215 may therefore serve as a patient reference sensor, providing a reference point, from which the position of the tracking sensor 210 may more accurately be estimated.

However, in other examples, the patient reference sensor 204 may be configured as an electromagnetic receiver, and the tracking sensor 210 may be configured as the electromagnetic transmitter. In such examples, the tracking sensor 210 may be supplied an initial current to generate the electromagnetic field, that may in turn be detected by the reference sensor 204. In yet further examples, the receiver sensor 211 may be configured as the electromagnetic transmitter. Thus, one of the reference sensor 204, tracking sensor 210, or receiver sensor 211 may be configured as an electromagnetic transmitter that generates an electromagnetic field when energized with an electric current.

The patient reference sensor 204 may include three coils arranged in an industry-standard coil arrangement (ISCA). Specifically, the patient reference sensor 204 may contain three approximately co-located, orthogonal quasi-dipole coils. However, in other examples more or less than three coils may be included in the patient reference sensor 204. Further, the orientation and/or arrangement of the coils included within the patient reference sensor 204 may be altered as desired. In some examples, the coils of patient reference sensor 204 may be concentrically positioned relative to one another. Further, the coils may be spaced approximately equally from one another about a center point.

Similar to the patient reference sensor 204, the tracking sensor 210 and receiver 215 may each include three primary coils. Specifically, the tracking sensor 210 and receiver 215 may each contain three approximately co-located, orthogonal quasi-dipole coils. However, in other examples more or less than three primary coils may be included in each of the tracking sensor 210 and receiver 215. The primary coils in each of the tracking sensor 210 and receiver 215 may be aligned substantially perpendicular to each other and may thus define a three-dimensional coordinate system. However, the orientation and/or arrangement of the primary coils included within the receivers 215 may be altered as desired. In some examples, the coils of tracking sensor 210 and receiver 215 may be concentrically positioned relative to one another. Further, the coils may be spaced approximately equally from one another about a center point.

The mutual inductances between each of the coils in the tracking sensor 210 and receiver 215, and each of the coils in the patient reference sensor 204 may be measured and/or estimated by the computing system 116. The position and orientation of the patient reference sensor 204 with respect to the tracking sensor 210 may then be calculated from the resulting mutual inductances of each of those coils and the knowledge of the coil characteristics. In this way, when the tracking sensor 210 is coupled to the medical instrument 212, the position of the medical instrument 212 may be estimated by the computing system 116 based on outputs from one or more of the tracking sensor 210 and receiver 215 and/or patient reference sensor 204.

As described in greater detail below with reference to FIGS. X-X, estimates of the position of the medical instrument 212 may be improved by calibrating the tracking sensor 210 based on the type of medical instrument 212 to which it is attached. The type of medical instrument 212 may include one or more of a manufacturer, item type, serial number, manufacturing date, supplier contact information, etc.

In some examples, the tracking sensor 210 may further include one or more magnetic field detectors, such as Hall effect sensors, for detecting a magnetic field signature of the medical instrument 212. The medical instrument 212 may include one or more magnets that generate a magnetic field. Each type of medical instrument 212 may include a unique pattern and/or arrangement of magnets that generate a distinct magnetic field. The magnets may be permanent magnets that generate their own persistent magnetic field. However, in other examples, the magnets may be electromagnets that produce a magnetic field when energized with an electrical current. In some examples, the medical instrument 212 may comprise two magnets. However, in other examples, more or less than two magnets may be included in the medical instrument 212. In this way, the medical instrument type may be determined based on the magnetic field signature of the medical instrument 212 via the Hall effect sensors of the tracking sensor 210. Specifically, the type of medical instrument 212 may be determined by the computing system 116 based on the magnetic field of the medical instrument 212 detected by the Hall effect sensors of the tracking sensor 210. However, in other examples, the medical instrument type may be determined via another identification method such as radio-frequency identification (RFID).

In some examples, one or more of the tracking sensor 210 and receiver 215 may be directly electrically coupled to the computing system 116 via one or more cables. However, in other examples, one or more of the tracking sensor 210 and receiver 215 may be wirelessly connected to the computing system 116 via wireless signals (e.g., via Bluetooth, Wifi, etc.). The tracking sensor 210 and receiver 215 may receive electrical power from the computing system 116. However, in other examples, one or more of the tracking sensor 210 and receiver 215 may receive electrical power from another source such as a wall socket, battery, generator, etc. In still further examples, one or more of the tracking sensor 210 and receiver 215 may include their own power source, such as a battery. Computing system 116 may monitor one or more of the current, voltage, and electrical power in the tracking sensor 210 and/or receiver 215 via the direct electrical connection.

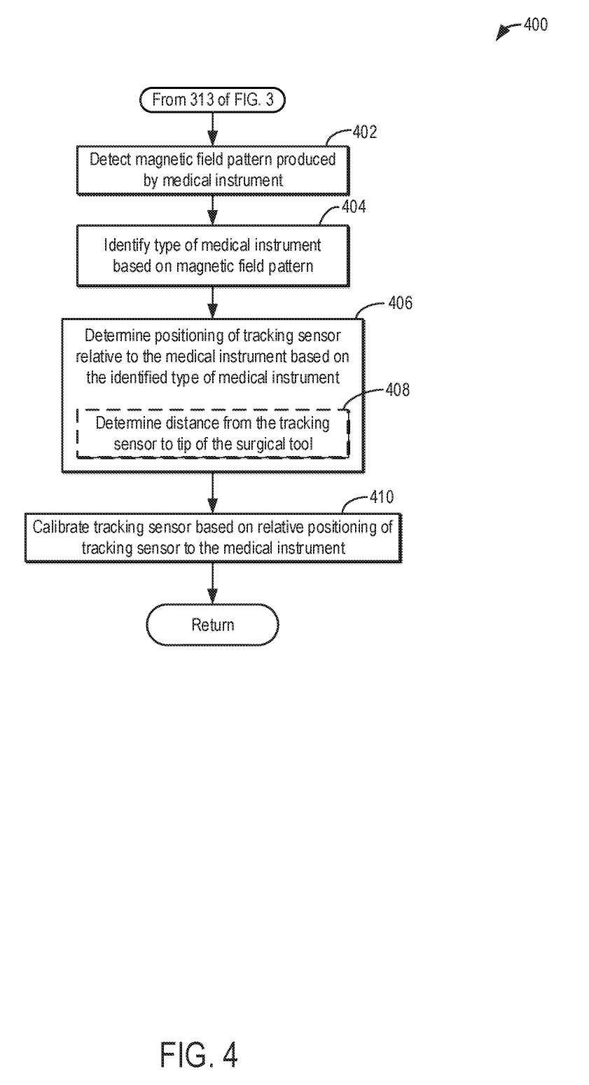

Computing system 116, may include the display screen 118, and a computing device 214. The computing device 214 includes various hardware and software components for executing instructions and control operations, such as those described in FIGS. 3-4. For example, the computing device 214 may include a logic subsystem 224, data-holding subsystem 218, image processing subsystem 220, and communication subsystem 222.

Logic subsystem 224 may include one or more processors that are configured to execute software instructions. For example, the logic subsystem 224 may include an image processor 220 for generating images of patient anatomy 108 and/or current positions of the medical instrument 212 based on instructions stored in data-holding subsystem 218 and outputs received from one or more of an X-ray detector (e.g., detector 112 shown in FIG. 1), patient reference sensor 204, and one or more of the tracking sensor 210 and receiver 215. Specifically, the image processor 220 may construct an image of the patient anatomy 108 based on outputs received from the X-ray detector. The image processor 220 may then construct images showing the relative positioning of the medical instrument 212 with respect to the patient anatomy 108 based on outputs from one or more of the tracking sensor 210 and/or receiver 215 and patient reference sensor 204.

Additionally or alternatively, the logic subsystem 224 may include one or more hardware or firmware logic machines configured to execute hardware or firmware instructions. Processors of the logic subsystem 224 may be single or multi-core, and the programs executed thereon may be configured for parallel or distributed processing. The logic subsystem 224 may optionally include individual components that are distributed throughout two or more devices, which may be remotely located and/or configured for coordinated processing.

Data-holding subsystem 218 may include one or more physical, non-transitory devices configured to hold data and/or instructions executable by the logic subsystem 224 to implement the herein described methods and processes. Thus, the methods and routines described below with reference to FIGS. 3-4 may be stored in non-transitory memory of data-holding subsystem 218. When such methods and processes are implemented, the state of data-holding subsystem 218 may be transformed (for example, to hold different data). Further, information such as look-up tables that may be used to implement the herein described methods and processes may be stored in non-transitory memory of the data-holding subsystem 218. For example, product information such as the size, weight, dimensions, length, volume, specifications, distance to tool tip, manufacturer etc., for each type of medical instrument 212 may be stored in non-transitory memory of the data-holding subsystem 218.

Data-holding subsystem 218 may include removable media and/or built-in devices. Data-holding subsystem 218 may include optical memory (for example, CD, DVD, HD-DVD, Blu-Ray Disc, etc.), and/or magnetic memory devices (for example, hard drive disk, floppy disk drive, tape drive, MRAM, etc.), and the like.

It is to be appreciated that data-holding subsystem 218 includes one or more physical, non-transitory devices. In contrast, in some embodiments aspects of the instructions described herein may be propagated in a transitory fashion by a pure signal (for example, an electromagnetic signal) that is not held by a physical device for at least a finite duration. Furthermore, data and/or other forms of information pertaining to the present disclosure may be propagated by a pure signal.

When included, communication subsystem 222 may be configured to communicatively couple the computing system 116 with one or more other computing devices. For example, the communication subsystem 222 may be configured to connect the computing device 214 with one or more of the tracking sensor 210 and receiver 215 and/or patient reference sensor 204. Communication subsystem 222 may include wired and/or wireless communication devices compatible with one or more different communication protocols. Thus, the communication subsystem 222 may communicatively couple the computing system 116 with one or more of the tracking sensor 210 and receiver 215, patient reference sensor 204, and an X-ray detector (e.g., detector 112 shown in FIG. 1) via a wireless or wired connection. As non-limiting examples, communication subsystem 206 may be configured for communication via a wireless telephone network, a wireless local area network, a wired local area network, a wireless wide area network, a wired wide area network, etc.

Turning now to FIG. 2B, it shows an alternate example of the tracking system 200 where the patient reference assembly 202 is physically coupled to the patient anatomy 108 and reference receiver sensor 211, described above in FIG. 2A, is not included. Thus, the reference receiver sensor 211 may be eliminated from the tracking system 200 in the example shown in FIG. 2B. As such, the patient reference assembly 202 may serve as the patient reference sensor in the example of the tracking system 200 shown in FIG. 2B.

In the example shown in FIG. 2B, mount 206 of patient reference assembly 202 may be physically coupled to bone 216 of the anatomy 108. However, in other examples, the mount 206 may be coupled to other components of the anatomy 108, such as tissue, muscle, blood vessels, fat, organs, etc.

Other than the positioning of the patient reference assembly 202 relative to the patient anatomy 108 and exclusion of the reference receiver sensor 211 however, FIG. 2A may be the same and/or identical to FIG. 2A. Thus, the only difference between FIGS. 2A and 2B may be the location at which the patient reference assembly 202 is coupled. As such, components of the tracking system 200 already introduced in the description of FIG. 2A may not be reintroduced or described again in the description of FIG. 2B herein.

In the example of FIG. 2B, the reference receiver sensor 211 described above with reference to FIG. 2A is not included in the tracking system 200. As such, the current position of the medical instrument 212 may be based on outputs from one or more of the patient reference sensor 204 and tracking sensor 210 only. Thus, in examples where the patient reference assembly 202 is coupled to the patient anatomy 108, the computing system 116 may estimate the current position of the medical instrument 212 based on outputs received from the tracking sensor 210 and/or patient reference sensor 204.

More specifically, the mutual inductances between each of the coils in the tracking sensor 210, and each of the coils in the patient reference sensor 204 may be measured and/or estimated by the computing system 116. The position and orientation of the transmitter of the tracking sensor 210 with respect to the patient reference sensor 204 may then be calculated from the resulting mutual inductances of each of those coils and the knowledge of the coil characteristics.

For example, when three coils are included in each of the patient reference sensor 204 and tracking sensor 210, the position and orientation of the transmitter tracking sensor 210 may be estimated based on nine resulting mutual inductances. However, in other examples, when more or less than three coils are used in the patient reference sensor 204 and tracking sensor 210, more or less than nine mutual inductances may occur.

Additionally, in examples where the patient reference assembly 202 is coupled to the patient anatomy 108, a secondary coil may be included in the tracking sensor 210 and/or reference sensor 204. The secondary coil may be oriented such that it is substantially non-parallel with each of the other coils of the sensor 210 or 204 in which it is included. Said another way, the secondary coil may be positioned such that its magnetic axis is not parallel with the magnetic axes of the other receiver coils of the sensor 210 or 204 in which it is included. The fourth coil may be used to resolve hemispherical ambiguity that can occur when using three-coil assemblies in the tracking sensor 210 and patient reference sensor 204. In this way, the computing system 116 may determine a current position of the tracking sensor 210 based on outputs received from one or more of the tracking sensor 210 and patient reference sensor 204.

In some examples, such as when the reference sensor 204 is configured as an electromagnetic transmitter and the tracking sensor 210 is configured as an electromagnetic receiver, the fourth coil may be included in the tracking sensor 210. In other examples, such as when the reference sensor 204 is configured as an electromagnetic receiver and the tracking sensor 210 is configured as an electromagnetic transmitter, the reference sensor 204 may include the fourth coil. In yet further examples, the fourth coil may be included in the tracking sensor 210 regardless of the configuration of the sensors 204 and 210 as transmitters or receivers.

Turning now to FIG. 3, it shows a flow chart of an example method 300 for displaying a position of a medical instrument (e.g., medical instrument 212 shown in FIGS. 2A and 2B) with respect to patient anatomy (anatomy 108 shown in FIGS. 1-2B) during image-guided surgery. A C-arm (e.g., C-arm 110 shown in FIG. 1) may be used to capture an image of the patient anatomy. During image-guided surgery the position of the medical instrument may be estimated using an electromagnetic tracking system (e.g., electromagnetic tracking system 200 shown in FIGS. 2A and 2B). Specifically, the position of the medical instrument may be estimated based on outputs from electromagnetic coil assemblies included within each of a tracking sensor (e.g., tracking sensor 210 shown in FIGS. 2A and 2B) and a patient reference sensor (e.g., patient reference sensor 204 shown in FIGS. 2A and 2B). Specifically, a first coil assembly may be included in the reference sensor of a patient reference assembly (e.g., patient reference assembly 202 shown in FIGS. 2A and 2B) and a second coil assembly may be included in the tracking sensor of a medical instrument assembly or tracking assembly (e.g., medical instrument assembly 208 shown in FIGS. 2A and 2B) of the tracking system.

One of either the tracking sensor or the patient reference sensor may be configured as an electromagnetic transmitter, while the sensor not configured as an electromagnetic transmitter may be configured as an electromagnetic receiver. Thus, in some examples, the tracking sensor may be configured as a receiver and the patient reference sensor may be configured as a transmitter. However, in other examples, the tracking sensor may be configured as a transmitter and the patient reference sensor may be configured as a receiver. An image of the patient anatomy, including the current position of the medical instrument with respect to the anatomy, may then be displayed to a surgeon or other medical personnel based on outputs from the patient reference sensor and tracking sensor.

Portions or all of method 300 may be stored in non-transitory memory (e.g., data-holding subsystem 218 shown in FIGS. 2A and 2B) of a computing device (e.g., computing device 214 shown in FIGS. 2A and 2B). As such, portions or all of method 300 may be executed by the computing device to display a position of the medical instrument relative to the patient anatomy to a medical operator (e.g., operator 106 shown in FIG. 1).

Method 300 begins at 302 which comprises securing a patient reference assembly mounting platform (e.g., mounting platform 206 shown in FIGS. 2A and 2B) to a patient (e.g., patient 102 shown in FIGS. 1-2). More specifically, the method 300 at 302 may comprise physically coupling the patient reference assembly mounting platform to an operating area (e.g., anatomy 108 shown in FIGS. 1-2) of the patient. Thus, the method at 302 comprises physically coupling the mounting platform of the patient reference assembly to the patient. The mount may be secured to the patient by one or more of screwing, clamping, and securing (e.g., via an adhesive) the mounting platform to the patient. However, other suitable mechanical/adhesive linkages may be used to secure the mounting platform to the patient. For example, the mounting platform may be threaded and may be secured to bone (e.g., bone 216 shown in FIGS. 2A and 2B) by screwing the mount into the bone. Specifically, the mounting platform may be coupled to the spine and/or Iliac Crest of the patient. However, in other examples, the mounting platform may be secured to another part of the patient, such as skin. In yet further examples, the mounting platform may be secured to a location external to the patient.

After securing the mounting platform of the patient reference assembly to the patient at 302, method 300 may then continue to 304 which comprises physically coupling the patient reference sensor to the patient reference assembly mounting platform. The patient reference sensor and the mounting platform may include mating mechanical interfaces that physically couple and decouple the two components when an external force is applied. For example, the patient reference sensor and mounting platform may include complementary mating elements that couple and decouple the patient reference assembly mounting platform and patient reference sensor when a force is provided by a surgeon or other medical personnel. Specifically, the patient reference sensor and patient reference assembly mounting platform may be coupled and decoupled from one another by rotating the two components relative to one another, thereby manipulating the mating elements. When the patient reference sensor and mounting platform are physically coupled to one another, the two components are attached and mechanically linked to one another, thus forming the patient reference assembly.

Method 300 may then proceed from 304 to 306 which comprises powering on an X-ray generator (e.g., generator 114 shown in FIG. 1) of the C-arm. In some examples, the method 300 at 306 may comprise holding the C-arm substantially stationary while powering on the X-ray generator. However, in other examples, the method 300 at 306 may comprise swiveling the C-arm a threshold number of degrees while powering on the X-ray generator. Thus, as described above with reference to FIG. 1, in some examples, the X-ray generator may be powered on while rotating the C-arm about a central rotational axis (e.g., axis X-X′ shown in FIG. 1). More specifically, in some examples, the method 300 at 306 may comprise swiveling the C-arm from a first position to a second position and powering on the X-ray generator for the duration of the movement from the first position to the second position. However, in other examples, the C-arm may be powered on for only a portion of the duration of the movement from the first position to the second position. Powering on the C-arm may comprise providing electrical power (e.g., voltage and current) to the C-arm.

In some examples, the threshold number of degrees may be approximately 180 degrees. However, in other examples, the threshold number of degrees may be greater or less than 180 degrees. In some examples, the threshold number of degrees that the C-arm may be rotated while powering on the X-ray generator may be determined based on surgical operating conditions such as a desired image quality, patient size and weight, patient anatomy, type of surgical operation, etc. However, in other examples, the threshold may be a preset number of degrees.

In examples where the C-arm is held substantially stationary while powering on the X-ray generator, the X-ray generator may be powered on for a threshold duration, and then after the duration, the X-ray generator may be turned off. However, in examples where the C-arm is rotated while powering on the X-ray generator, the X-ray generator may be turned off once the C-arm has been rotated the threshold number of degrees, or has completed its rotation. That is, electrical power provided to the X-ray generator may be terminated.

While the X-ray generator is powered on at 306, an X-ray detector (e.g., detector 112 shown in FIG. 1) of the C-arm may detect X-rays produced by the X-ray-generator at 308. Thus, the method 300 at 308 may comprise receiving X-ray produced by the X-ray generator. In this way, 306 and 308 may be executed approximately simultaneously. As described above with reference to FIG. 1, the X-ray detector may convert the received X-ray intensity into a digital output.

After the X-ray generator is powered off at 306, and the X-rays are received at 308, method 300 may then proceed to 310 which comprises generating a first image of the patient anatomy based on the received X-rays. Specifically, as described above with reference to FIG. 1, an image of the patient anatomy may be generated based on the relative intensities of X-rays received at the received at the X-ray detector at different points along the detection medium (e.g., surface 113 shown in FIG. 1). In some examples, such as examples where the C-arm is held substantially stationary when powering on the X-ray generator at 306, the first image may be a two dimensional image. However, in other examples, such as examples where the C-arm is rotated when powering on the X-ray generator, the first image may be a three dimensional image that may be compiled based on the X-rays received from different angles and positions during the rotation of the C-arm. In such examples, the method 300 at 310 may comprising compiling outputs received from the detector during the rotation of the C-arm while the X-ray generator was powered on, into a three dimensional image.

Method 300 may then continue from 310 to 312 which comprises physically coupling the tracking sensor to a medical instrument (e.g., medical instrument 212 shown in FIGS. 2A and 2B). Similar to the transmitter and patient reference assembly mounting platform, the tracking sensor and medical instrument may include mating mechanical interfaces that physically couple and decouple the two components when an external mechanical force is applied. For example, as described in greater detail below with reference to FIGS. 5-17, one or more of the tracking sensor and/or medical instrument may include an adjustable snapping latch (e.g., attachment device 726 described below in FIGS. 7-17). The tracking sensor and medical instrument be coupled and decoupled from one another by manipulating the snapping latch.

Specifically, the snapping latch may be pivotable, and thus the latch may be pivoted via a mechanical torqueing force to couple and/or decouple the tracking sensor and medical instrument. In some examples, as described below with reference to FIGS. 8-9, 11-12, 14-15, and 17, the tracking sensor may be coupled to a handle (e.g., handle 704 shown in FIGS. 7-9, 11-12, 14-15, and 17) of the medical instrument. When the medical instrument and tracking sensor are physically coupled to one another, the two components are attached and mechanically linked to one another, thus forming the medical instrument assembly. However, when decoupled from one another, the medical instrument and tracking sensor may form two distinct components that do not physically contact one another.

In some examples, the method 300 at 312 may comprise providing a notification to an operator when the tracking sensor becomes coupled to the medical instrument. The notification may be passive and/or active. For example, as described in greater detail below with reference to FIGS. 8-17, the latch may provide a noise, such as an auditory “click” when the tracking sensor is coupled to the medical instrument. However, in other examples, the operator may be actively notified when the tracking sensor is successfully coupled to the medical instrument. For example, the medical instrument may include a user input device (e.g., user input device 524 described in greater detail below with reference to FIG. 5) that may one or more of change color, vibrate, produce sounds, illuminate, etc., in response to coupling of the tracking sensor to the medical instrument.

After coupling the tracking sensor and medical instrument at 312, method 300 then proceeds to 313 which comprises determining the medical instrument type based on a magnetic signature of the medical instrument. The medical instrument may include one or more magnets that generate a magnetic field. As described in greater detail below with reference to FIGS. 5 and 17 the tracking sensor may include Hall effect sensors for detecting the magnetic field generated by the magnets in the medical instrument. The medical instrument type is determined based on the magnetic field detected by the Hall effect sensors. Thus, the tool type may be estimated based on outputs from the Hall effect sensor.

However, it should be appreciated that in other examples, another type of product identification method may be employed. For example, the medical instrument type may be determined using radio-frequency identification (RFID).

In some examples, each type of medical instrument may comprise its own unique pattern and/or arrangement of magnets, such that the magnetic field produced by each type of medical instrument may be different. Said another way, each medical instrument type may include its own unique magnetic field signature, that may be used to identify that tool. The medical instrument type may include one or more of a name, product type, manufacturer, brand, product identification number, serial number, manufacturing date, supplier contact information, etc.

Further, the method 300 at 312 may include determining product information for the medical instrument to which the tracking sensor is coupled based on the tool type. The dimensions, specifications, and other product information for each specific tool type may be known and/or stored in non-transitory memory. As such, product information including one or more of the size, weight, dimensions, distance to tool tip, specifications, etc., for the tool to which the tracking sensor is coupled may be determined from a look-up table/stored memory based on the tool type. In some examples, the product information may be stored in the non-transitory memory of the computing device.

Further, the method 300 at 312 may comprise calibrating the tracking sensor based on the medical instrument type. Thus, after determining the type of medical instrument the tracking sensor is coupled to, the method 300 may comprise calibrating the tracking sensor based on the dimensions of the medical instrument, and a known position of the tracking sensor relative to the medical instrument when coupled together. For example, distances between the tracking sensor and tips of each type of medical instrument when the tracking sensor is coupled thereto may be known and may be stored in a look-up table. As such, the distance between the tracking sensor and the tip of the medical instrument to which the tracking sensor is coupled may be determined based on the medical instrument type, and a known distance between the tracking sensor and that type of medical instrument when the two are coupled together.

After determining the medical instrument type at 313 and calibrating the tracking sensor, method 300 may continue to 314 which comprises powering on the transmitter and producing electromagnetic signals. As described above, either the tracking sensor or patient reference sensor may be configured as the transmitter. Thus, the method 300 at 314 may comprise providing electrical power to either the tracking sensor or the patient reference sensor. Specifically, powering on the transmitter may comprise flowing current through the electromagnetic coils included in the transmitter. Thus, the method 300 at 314 may comprise providing electrical power (e.g., voltage and/or current) to the transmitter. By powering on the transmitter, the transmitter may generate an electromagnetic field and produce electromagnetic radiation or waves. In some examples, the electromagnetic waves may be radio waves.

After powering on the transmitter at 314, the method 300 may then continue to 315 which comprises receiving the electromagnetic signals from the transmitter. As explained above with reference to FIG. 2A and 2B, powering on the transmitter and generating an electromagnetic field may induce current to flow in the electromagnetic coils of the receiver (mutual inductance). Thus, the electromagnetic signals generated by the transmitter may be received by the receiver at 315. Further, the induced current flow in the receiver, may correspondingly cause changes in the electrical current of the coils of the transmitter due to mutual inductance between the transmitter and receiver. Thus, the outputs (e.g., current and/or voltage) from the transmitter and the receiver may be affected and/or changed due to the current supplied to the transmitter. As the position and/or orientation of the transmitter relative to the receiver changes, the outputs from the transmitter and receiver may change.

The method 300 may then continue from 315 to 316 which comprises analyzing the outputs from the transmitter (e.g., patient reference sensor of the patient reference assembly) and receiver and determining the current position of the medical instrument based on the outputs. Thus, the method 300 at 316 comprises analyzing the outputs from the patient reference sensor and tracking sensor. As explained above, the outputs may be in the form of a voltage and/or electrical current. Thus, the method 300 at 316 may first comprise estimating a position of the receiver relative to the transmitter based on the outputs received from the transmitter and the receiver. However, in other examples, the method at 316 may comprising estimating a position of the transmitter relative to the receiver based on the outputs received from the transmitter and receiver. Then, a current position and/or orientation of the medical instrument may be estimated based on known geometric transformation relating the position of the receiver or transmitter (whichever is coupled to the medical instrument) to a position of the medical instrument. More simply, the outputs received from the transmitter and receiver may be calibrated to determine the current position of the medical instrument. However, it should be appreciated that in other examples, the position of the transmitter or receiver may be estimated based on outputs from either the transmitter or the receiver, and that outputs from both the transmitter and receiver may not be used to estimate the position of one relative to the other.

Based on the current position of the medical instrument determined at 316, method then proceeds to generate a second image at 318. The second image may be an image of the medical instrument overlaid onto the first image. Specifically, based on the known size, and dimensions of the medical instrument, an image of the medical instrument may be constructed based on the current position of the medical instrument estimated at 316, where the current position of the medical instrument may be determined based on the estimated positions of the receiver and/or transmitter relative to one another. Thus, the second image may show the current position of the medical instrument relative to the patient anatomy. In this way, both the patient anatomy, and position of the medical instrument may be estimated.

It is important to note that 314, 316, and 318 may be executed approximately continuously while the transmitter is powered on. Thus, the method 300 may return back to 315 and continue to determine the current position of the medical instrument as long as the transmitter remains powered on. Thus, the position of the medical instrument may be updated based on the most recent outputs received from one or more of the transmitter and receiver to reflect the most recent position of the medical instrument. In this way, estimates of the position of the medical instrument may be updated approximately continuously. However, in other examples, estimates of the position of the medical instrument may be updated at regular time intervals or after a pre-set duration has expired since a most recent estimate.

After generating the second image at 318, method 300 may continue to 320 which comprises displaying the second image to the medical operator via a display screen (e.g., display screen 118 shown in FIGS. 1-2B). Thus, the method 300 at 318 comprises displaying an image of the patient anatomy, with a current or more recent position of the medical instrument. Thus, a visual representation of the patient anatomy and medical instrument position may be presented to the medical operator in either a two dimensional or three dimensional image. Method 300 then returns.

As explained above at 306, the C-arm may be rotated to acquire an image of the patient anatomy from a different vantage point. However, in some examples, the C-arm may be rotated while the transmitter is powered on and the position of the medical instrument is being estimated. Thus, in some examples, 306 may be executed after powering on the transmitter. For example, the method 300 at 320 may comprise rotating the C-arm and powering on the C-arm for a duration to acquire a new image of the patient anatomy. In some examples, multiple X-ray images from different angles may be combined to create three dimensional images of the patient anatomy.

Turning now to FIG. 4, it shows a flow chart of an example method 400 for identifying a type of medical instrument (e.g., medical instrument 212 shown in FIGS. 2A and 2B). Specifically method 400 may be a subroutine of method 300 described above with reference to FIG. 3, and may be executed at 313 of method 300. Thus, method 400 may be executed after physically coupling a tracking sensor (e.g., tracking sensor 210 shown in FIGS. 2A and 2B) to the medical instrument to determine the medical instrument's type. The type of medical instrument may be determined based on a magnetic field signature produced by permanent magnets included in the medical instrument. Based on the determined type of medical instrument, the tracking sensor may be calibrated to obtain more accurate estimates of the position of the medical instrument.

Portions or all of method 400 may be stored in non-transitory memory (e.g., data-holding subsystem 218 shown in FIGS. 2A and 2B) of a computing device (e.g., computing device 214 shown in FIGS. 2A and 2B). As such, portions or all of method 400 may be executed by the computing device to identify the medical instrument type, and adjust estimates of the medical instrument position based on the instrument type.

Method 400 begins at 402 which comprises detecting a magnetic field pattern produced by the medical instrument. Specifically, as described above with reference to FIGS. 2A and 2B, magnets such as permanent magnets may be included in the medical instrument. The method at 402 comprises detecting the magnetic field produced by these magnets. The magnetic field may be detected via one or more magnetic field detectors, such as Hall effect sensors (e.g., Hall effect sensors 552 shown in FIGS. 5 and 17) included in the tracking sensor. Thus, based on outputs from the magnetic field detectors, a magnetic field pattern produced by the medical instrument may be detected at 402.

Specifically, as described in greater detail below with reference to FIGS. 5 and 17, each magnetic field detector may sense either a north, south, or no magnetic field. Further, each of the magnetic field detectors may be spatially separated from one another. In this way, the presence and/or polarity of a magnetic field may be detected at each physical location of the magnetic field detectors. In some examples, the strength of the magnetic field may also be detected. In this way, a magnetic field pattern generated by the medical instrument may be estimated based on the collective outputs from the magnetic field detectors.

The number of unique, detectable magnetic field patterns, may be increased by including more magnetic field detectors. Additionally, the detection area may be adjusted by changing the distance between magnetic field detectors. For example, the detection area may be increased by increasing the distance between detectors. Further, the spatial resolution of the estimated magnetic field pattern may be adjusted by adjusting the number and/or density of the magnetic field detectors within the detection area.

After detecting the magnetic field pattern at 402, method 400 may then continue to 404 which comprises identifying the type of medical instrument based on the magnetic field pattern detected at 402. As explained above with reference to FIGS. 2A-3, each type of medical instrument may comprise its own unique magnetic field pattern. That is, the number, strength, and/or orientation of the magnets included in the medical instrument may be different for each type of medical instrument, such that the magnetic field produced by each type of medical instrument, and therefore the combination of outputs received from the magnetic field detectors is specific to a particular type of medical instrument. Said another way, the magnetic field pattern detected via the magnetic field detectors may be unique for each type of medical instrument. Thus, based on the combination of outputs from the magnetic field detectors, a type of medical instrument may be determined at 404. As described above with reference to FIGS. 1-3, the medical instrument may comprise one or more of forceps, clamps, retractors, distractors, scalpels, lancets, dilators, suction tips and tubes, injection needles, drills, endoscopes, tactile probes, biopsy needle, pin, scope, jamshidi, drill, drill guide, screwdriver etc.

Method 400 may then continue from 404 after identifying the type of medical instrument to 406, which comprises determining the positioning of the tracking sensor relative to the medical instrument based on the identified type of medical instrument. As explained above with reference to FIG. 3, the medical instrument product information may be retrieved based on the type of medical instrument. Thus, the product information for the specific type of medical instrument to which the tracking sensor is coupled may be obtained. For example, the product information may be stored in a look-up table in non-transitory memory, and may be retrieved based on the identified type of medical instrument. The product information may include one or more of the dimensions, specifications, length, size, weight, manufacturer, name, product identification number, serial number, color, distance to tool tip, etc., of the medical instrument. Thus, the product information for each type of medical instrument may specify the relative positioning of the tracking sensor and medical instrument when the tracking sensor and medical instrument are coupled to one another.

As such, the method at 406 may comprise determining the distance from the tracking sensor to each point along the perimeter or edge of the medical instrument. For example, the method 400 at 406 may include determining the distance from the tracking sensor to a tip (e.g., tip 707 shown in FIG. 7) of the medical instrument at 408. In this way, an outline, perimeter, or image of the medical instrument may be estimated based on the position of the tracking sensor, and the known positioning of the tracking sensor relative to the medical instrument. The relative positioning of the tracking sensor and medical instrument may be different for each type of medical instrument.

Method 400 may proceed from 406 to 410 which comprises calibrating the tracking sensor based on the relative positioning of the tracking sensor to the medical instrument. Thus, outputs from the tracking sensor may be adjusted based on the type of medical instrument to which the tracking sensor is coupled, and the known relative positioning of the tracking sensor relative to that type of tool. In this way, more accurate estimates of the position of the medical instrument may be achieved by identifying the type of medical instrument to which the tracking sensor is coupled, and using known information regarding the dimensions of that type of instrument. Method 400 then returns.

Turning now to FIGS. 5-19 they depict embodiments of an example electromagnetic tracking sensor (e.g., tracking sensor 210 described above in FIGS. 2A and 2B) used in an electromagnetic surgical navigation system (e.g., electromagnetic tracking system 200 described above in FIGS. 2A and 2B) during image guided surgery. Further, FIGS. 5-17 show example attachment mechanisms, for coupling the tracking sensor to a medical instrument (e.g., medical instrument 212 shown in FIGS. 2A and 2B). Specifically, the tracking sensor may be removably coupled to a handle (e.g., handle 704 shown in FIGS. in FIGS. 7-9, 11-12, 14-15, and 17) of the medical instrument. When coupled to one another, the tracking sensor and medical instrument may form a medical instrument assembly (e.g., medical instrument assembly 208 shown in FIGS. 2A and 2B).

More specifically, the medical instrument may include an interface having a first mating surface, included within the handle of the medical instrument. Further, the electromagnetic tracking sensor may include a second mating surface, and the tracking sensor may be removably coupled to the medical instrument via the first and second mating surfaces. An attachment device included on the first mating surface and/or second mating surface, may be adjusted to physically couple and decouple the electromagnetic tracking sensor and medical instrument via the first and second mating surfaces. The attachment device may be one or more of a hinge, pivotable flange or tab, groove, pin, or other mechanical attachment mechanism.

FIGS. 5-9 depict an example embodiment, where the attachment device of the medical instrument assembly is included on the handle of the medical instrument. Specifically, the attachment device may be configured as a pivotable flange within the handle of the medical instrument. However, in an alternate embodiments shown in FIGS. 10-17, the attachment device may be included on the tracking sensor. Specifically, FIGS. 10-12 show an example embodiment of the medical instrument assembly, where the attachment device is configured as a pivotable tab that extends from an end of the tracking sensor. In other embodiments shown in FIGS. 13-15, the tracking sensor may include two overhangs that permit rotation of the tracking sensor relative to the handle of the medical instrument. Further, FIGS. 16-17 show an example embodiment of the medical instrument assembly where the tracking sensor includes a heel and pivotable tab positioned at opposite ends of the tracking sensor.

Similar components of the medical instrument assembly may be shown throughout FIGS. 5-19. Thus, components of the medical instrument assembly (also referred to herein as instrument tracking assembly) that are labeled similarly in FIGS. 5-19 may only be introduced and described once, after which they may not be reintroduced or described again. Further, FIGS. 5-19 may be described together in the description herein.

FIGS. 5-19 may be drawn approximately to scale, and thus show relative sizing and positioning of components of the medical instrument assembly with respect to each other. However, it should be appreciated that in alternative embodiments, different relative sizing and/or positioning may be used, if desired. FIGS. 5-19 show, for reference, an axis system 507 displaying a vertical axis 501, horizontal axis 503, and lateral axis 505. Components positioned or described to be above, are in the positive direction of the vertical axis 501. Further components positioned or described to be below are in the negative direction of the vertical axis 501. Similarly, components described as being in front may be in the negative direction of the lateral axis 505, and components described as being behind may be in the positive direction of the lateral axis 505.

Further, FIGS. 5-17 show the relative positioning of various components of the medical instrument assembly. If shown directly contacting each other, or directly coupled, then such components may be referred to as directly contacting or directly coupled, respectively, at least in one example. Similarly, components shown contiguous or adjacent to one another may be contiguous or adjacent to each other, respectively, at least in one example. As an example, components lying in face-sharing contact with each other may be referred to as in face-sharing contact or physically contacting one another. As another example, elements positioned apart from each other with only a space there-between and no other components may be referred to as such, in at least one example.

As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

Turning now to FIGS. 5-9, they show examples of a first embodiment of the medical instrument assembly, where the first mating interface of the handle of the medical instrument includes the attachment device.

Focusing on FIG. 5, it shows an exterior side perspective view 500 of an example tracking sensor 502 (e.g., tracking sensor 210 described above in FIGS. 2A and 2B). Thus, tracking sensor 502 may be the same or similar to tracking sensor 210 described above with reference to FIGS. 2A and 2B. As such, tracking sensor 502 may be included in the electromagnetic surgical navigation system (e.g., electromagnetic tracking system 200 shown in FIGS. 2A and 2B).

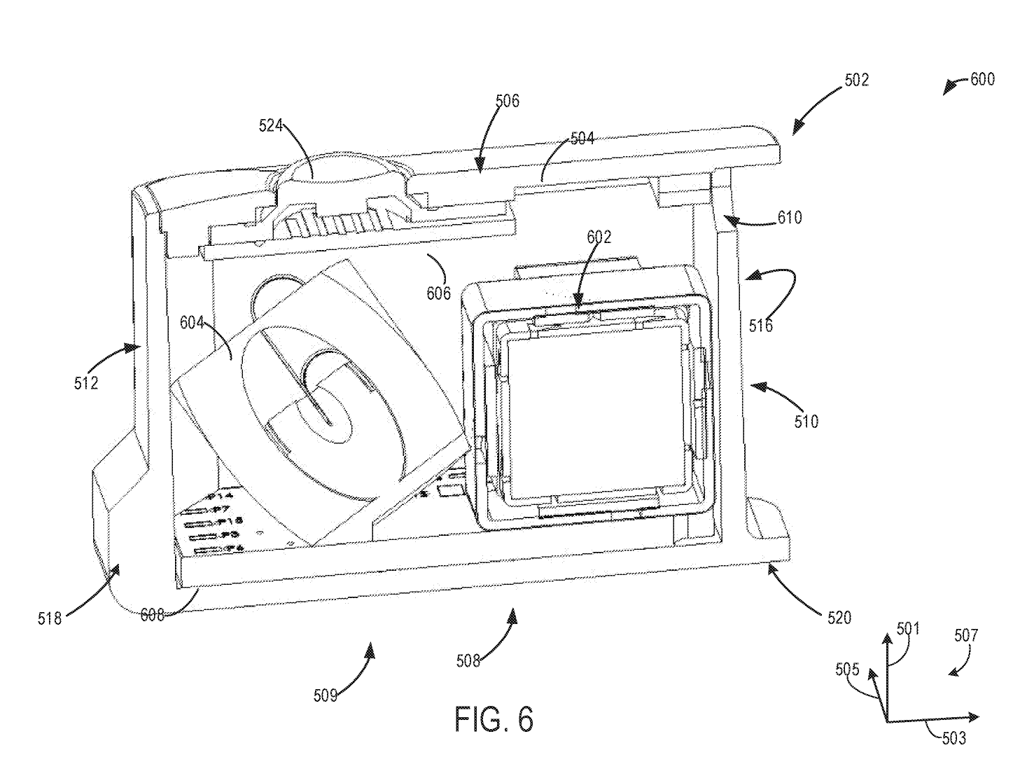

Tracking sensor 502 comprises a housing 504 that encloses interior components of the tracking sensor 502, which will described in greater detail below with reference to FIG. 6. The housing 504 may include six walls: top wall 506, bottom wall 508, first side wall 510, second side wall 512, front wall 514, and back wall 516. The walls 506, 508, 510, 512, 514, and 516 of the housing 504 may be fluidically sealed with one another along their edges, and thus may seal interior and exterior portions of the tracking sensor 502 from one another. In this way, the housing 504 may define a volume of the tracking sensor 502. The top wall 506 may be positioned opposite the bottom wall 508, defining a height of the tracking sensor 502. Further, the housing includes the first side wall 510 opposite the second side wall 512, defining a length of the tracking sensor 502. The front wall 514 may be positioned opposite the back wall 516, which is not visible in FIG. 5, defining a width of the tracking sensor 502.

A first mating surface of the handle of the medical instrument interfaces with and/or physically contacts a second mating surface 509 of the tracking sensor 502. Thus, the second mating surface 509 interfaces with and/or physically contacts the first mating surface of the tracking sensor interface of the of the handle of the medical instrument to physically couple and decouple the tracking sensor 502 and medical instrument. Second mating surface 509 may include the bottom wall 508, and a portion of one or more of the first side wall 510, second side wall 512, front wall 514, and back wall 516.

The second side wall 512 may include a stabilizing heel 518 that protrudes from the surface of the second side wall 512 and may form a portion of the second mating surface 509. Thus, the second side wall 512 may be approximately flat and/or planar other than the stabilizing heel 518. In some examples, as shown in the example of FIG. 5, the stabilizing heel 518 may extend from the bottom wall 508, up a portion of the side wall 512. Further, the stabilizing heel 518 may extend between the front wall 514 and back wall 516. However, in other examples, the stabilizing heel 518 may only extend along a portion of the width of the tracking sensor 502, and may not extend between the front wall 514 and back wall 516. The stabilizing heel 518 may help retain the position of the tracking sensor 502 relative to the medical instrument when coupling the tracking sensor 502 to the medical instrument. Specifically, the stabilizing heel 518 may engage a ledge (e.g., ledge 730 described below in FIG. 7) of the first mating surface of the tracking sensor interface.

The first side wall 510 may include a latching lip 520 that protrudes from the surface of the first side wall 510 and may from a portion of the second mating surface 509. Thus, the first side wall 510 may be approximately flat and/or planar other than the latching lip 520. In some examples, as shown in the example of FIG. 5, the latching lip 520 may extend from the bottom wall 508, up a portion of the side wall 512. Further, the latching lip 520 may extend between the front wall 514 and back wall 516. However, in other examples, the latching lip 520 may only extend along a portion of the width of the tracking sensor 502, and may not extend between the front wall 514 and back wall 516. The latching lip 520 may engage with an attachment device (e.g., flange 726 described below in FIG. 7) of the handle of the medical instrument to couple the tracking sensor 502 to the medical instrument.

Thus, one or more of the heel 518, lip 520, and walls 508, 510, 512, 514, and 516 may form the second mating surface 509 that interfaces with the second mating surface of the tracking sensor interface of the handle of the medical instrument.

Front wall 514 may further include one or more magnetic field detectors 522. For example, the magnetic field detectors 522 may be Hall effect sensors. However, in other examples, the magnetic field detectors 522 may be any other suitable sensors for detecting a magnetic field. Although depicted in the example of FIG. 5 to be positioned on the front wall 514, it should be appreciated that the magnetic field detectors 522 may be positioned elsewhere on or within the tracking sensor 502.

For example, the magnetic field detectors 522 may be positioned on the bottom wall 508. However, in other examples, the magnetic field detectors 522 may not be positioned on any of the walls 506, 508, 510, 512, 514, and 516, and may instead be positioned within the housing 504 of the tracking sensor 502, internal to the walls 506, 508, 510, 512, 514, and 516. Thus, although depicted, positioned on an exterior surface of the housing 504, it is important to note that the magnetic field detectors 522 may not be visible on the exterior of the housing 504, and may be included as internal components of the tracking sensor 502. For example, the magnetic field detectors 522 may be positioned on an internally facing surface of one of the walls 506, 508, 510, 512, 514, and 516. In other examples, the magnetic field detectors 522 may be positioned adjacent to one of the internally facing surface of one of the walls 506, 508, 510, 512, 514, and 516, or proximate to, but not physically contacting one of the walls. For example, FIG. 17 shows an embodiment where the magnetic field detectors 522 are positioned adjacent to an internally facing surface of the bottom wall 508.

In the example of FIG. 5, exactly six magnetic field detectors 522 may be included in the tracking sensor 502. However, in other examples, more or less than six detectors 522 may be included in the tracking sensor 502. The detectors 522 may be arranged in rows and columns along a plane approximately parallel to one of the walls 506, 508, 510, 512, 514, 516, and 518. Specifically, the detectors 522 may be arranged in rows and columns along a plane parallel to front wall 514. However, the detectors 522 may be arranged in rows and columns along a plane parallel to bottom wall 508 as shown in the example of FIG. 17. Specifically, the detectors 522 may be arranged in three columns and two rows. However, in other examples, the number rows may be more or less than two and the number of column may be more or less than three.

It should be appreciated that in other examples, the magnetic field detectors 522 may be arranged in alternate orientations, patterns, or positions, such as any geometric or non-geometric distributions such as Gaussian. The distance between detectors 522, and detector density may be also be adjusted as desired.

Each of the detectors 522 may sense the presence of a magnetic field and/or a polarity (e.g., north or south) of a magnetic field when exposed to a magnetic field. As such, outputs from each of the detectors 522 may indicate whether or not a magnetic field is detected, and if a magnetic field is detected, whether the magnetic field is a north or south pointing magnetic field. Thus, each of the detectors 522 may generate one of three outputs that indicate: no magnetic field, south pointing magnetic field, or north pointing magnetic field. In this way, approximately (₁³)⁶ or 729 unique combinations of outputs from the detectors 522 may be obtained, when six detectors 522 are included in tracking sensor 502. As such, approximately 729 different magnetic field patterns may be detected by the magnetic field detectors 522. However, the number of different magnetic field patterns identifiable by the detectors 522 may be adjusted by including more or less magnetic field detectors 522.

Top wall 506 may include a user input device 524. As depicted in the example of FIG. 5, the user input device 524 may be a button, and thus user input device 524 may also be referred to in the description herein as user input button 524. However, in other examples, the user input device 524 may be a joystick, mouse, keyboard, touch screen display, or other type of user interface device. Further, the user input device 524 may include more than one button. The user input device 524 may provide a plurality of functionalities to an operator performing surgery (e.g., operator 106 shown in FIG. 1). For example, the user input device 524 may permit the operator to adjust operation of one or more of the tracking sensor 502 and/or medical instrument (e.g., medical instrument 702 described below in FIGS. 7-9, 11-12, 14-15, and 17).

Specifically, the user input device 524 may be manipulated and/or adjusted to adjust an operational setting of the tracking sensor 502 and/or medical instrument, power on and power off the tracking sensor 502 and/or medical instrument, save trajectory routes of the tracking sensor 502 and/or medical instrument, save anatomical features of a patient (e.g., patient 102 shown in FIGS. 1-2B), etc. The user input device 524 may be pressed, pushed, flipped, rotated, pivoted, etc.

Further, the input device 524 may provide tactile and/or visible feedback to the operator. For example, the input device 524 may change color, and/or vibrate. In one example, the input device 524 may change color and/or vibrate in response to the tracking sensor 502 and medical instrument being coupled together. Thus, the input device 524 may provide an indication or verification that the tracking sensor 502 and medical instrument are coupled to one another. In still further examples, the tracking sensor 502 may provide an auditory click that indicates that the tracking sensor 502 is coupled to the medical instrument.

In yet further examples, the operator may interact with a display screen (e.g., display screen 118 shown in FIGS. 1-2B) via manipulation of the user input device 524. For example, the input device 524 may allow the operator to toggle through and select from menu options on the display screen. Thus, the operator may select an alternate instrument type via the user input device 524 if the type of instrument to which the tracking sensor 502 is coupled is misidentified by the magnetic field detectors 524. Additionally, the user input device 524 may allow an operator to select an operating mode of the tracking sensor 502, recalibrate the tracking sensor 502, select a type of medical procedure, etc.

Additionally, the user input device 524 may include lights for indicating one or more of an operating mode of the tracking sensor 502, a power state of the receiver, and physical coupling of the receiver and the medical instrument. For example, the lights may be LED.

User input device 524 may be included on the top wall 506, more proximate the second side wall 512 than the first side wall 510 as depicted in FIG. 5. However, the positioning of the user input device 524 within the top wall 506 may be adjusted as desired. For example, the user input device 524 may be positioned nearer the first side wall 510 than the second side wall 512. Further, the user input device 524 may be included on another one of the walls 508, 510, 512, 514, and 516. In yet further examples, more than one user input device 524 may be included on any one or more of the walls of the housing 504. Cutting plane 525 defines a cross-section of the tracking sensor 502 shown in FIG. 6.

FIG. 6 shows a cross-section view 600 of the tracking sensor 502 taken along cutting plane 525 shown above in FIG. 5, exposing the interior components of the tracking sensor 502. As described above with reference to FIGS. 2A and 2B, the tracking sensor 502 includes a primary coil assembly 602 comprising an industry-standard coil arrangement (ISCA). Specifically, the primary coil assembly 602 comprises three approximately co-located, orthogonal quasi-dipole coils. Thus, coils may be arranged perpendicular to one another, forming three orthogonal planes that define a three dimensional coordinate system. However, in other examples more or less than three coils may be included in the primary coil assembly 602. Further, the orientation and/or arrangement of the coils included within the assembly 602 may be altered as desired.

Further, as described in detail above with reference to FIG. 2B, the tracking sensor 502 may include a secondary coil assembly 604. The secondary coil assembly 604 may comprise a single coil that is orientated such that it is substantially not parallel to any of the coils in the primary coil assembly 602. The secondary coil assembly 604 may be used in methods for reducing or eliminating hemisphere ambiguity that may arise when using three coils in the primary coil assembly 602 and in a patient reference sensor (e.g., patient reference sensor 204 shown in FIGS. 2A and 2B). The tracking sensor 502 may be configured as either an electromagnetic transmitter or receiver. In examples where a patient reference sensor is configured as an electromagnetic transmitter, the tracking sensor 502 may be configured as a receiver. However, in other examples where the patient reference sensor is configured as an electromagnetic receiver, the tracking sensor 502 may be configured as the transmitter.

For example, signals from each of the coils of the primary coil assembly 602, and coil assembly of the electromagnetic transmitter may be calculated and these signals may be processed with an analytical model to obtain solutions for two possible tracking sensor positions that may occur in opposite hemispheres. A signal from the fourth coil of the secondary coil assembly 604 may be used to determine the correct tracking sensor position. For the two possible tracking sensor positions, a magnetic field and a sensor coil model may provide the expected receiver voltage signals from each position. However, the position and/or orientation of the fourth coil of the secondary coil assembly 604 is different from that of the other three coils of the primary coil assembly 602 and may be asymmetric with respect to the coils of the patient reference sensor. As a result, the model-predicted signals from the fourth coil may differ between the two hemispheres. The correct hemisphere may be identified as the one that provides the closest match between the measured and the model-predicted signal for the fourth coil.

Continuing with the description of FIG. 6, the user input device 524 may be electrically and/or mechanically coupled to a first circuit board 606. The circuit board 606 may execute and/or store software instructions. Further, the first circuit board 606 may be a printed circuit board, that coverts mechanical movement of the user input device 524 into digital signals that may be stored in non-transitory memory. For example, in response to a movement of the user input device 524, the circuit board 606 may send signals to an image processor (e.g., image processor 220 described in FIGS. 2A and 2B) to change an output displayed on the display screen. In some examples, the first circuit board 606 may be mechanically attached to an internal surface of the top wall 506.

Further, the tracking sensor 502 includes a second circuit board 608 that may be physically coupled to and/or in face sharing contact with in interior surface of the bottom wall 508. Circuit board 608 may execute software instructions for sending signals from the tracking sensor 502 to a computing system (e.g., computing system 116 shown in FIGS. 1-2B), and/or adjusting operation the tracking sensor 502 based on signals received from sensors of the tracking sensor 502 and/or from the computing system. Further, the circuit board 608 may analyze signals received from the sensors and/or computing system.

The magnetic field detectors 522 (not shown in FIG. 6) described above with reference to FIG. 5, may be included in or on the second circuit board 608. Thus, the detectors 522 may be physically and/or electrically coupled to the second circuit board 608. Specifically, the detectors 522 may be coupled to a bottom surface of the circuit board 608, between the internal surface of the bottom wall 508 of the housing 504, and the circuit board 608.

The tracking sensor 502 may additionally include an opening 610 positioned at the first side wall 510 that may be sized to receive one or more electrical cables (e.g., cables 226 described above in FIGS. 2A and 2B). The opening 610 may be positioned more proximate the top wall 506 than the bottom wall 508. However, the opening 610 may be positioned at another location on the first side wall 510. It should also be appreciated that in other examples, the opening 610 may be positioned on one of the other walls 506, 508, 512, 514, or 516.

Moving on to FIG. 7, it shows a top perspective view 700 of an example medical instrument 702 (e.g., medical instrument 212 described above in FIGS. 2A and 2B) to which the tracking sensor 502 (not shown in FIG. 7) may be attached. Thus, medical instrument 702 may be the same or similar to medical instrument 212 described above in FIGS. 2A and 2B. As such, medical instrument 702 may be included in the electromagnetic surgical navigation system (e.g., electromagnetic tracking system 200 shown in FIGS. 2A and 2B). The medical instrument 702 and tracking sensor 502 may be removably coupled to one another via mating interfaces on each of the tracking sensor 502 and medical instrument 702. When coupled together, the tracking sensor 502 and medical instrument 702 may form a medical instrument assembly (e.g., medical instrument assembly 208 described in FIGS. 2A and 2B).

Medical instrument 702 comprises a handle 704 and a surgical tool 705. The handle 704 and surgical tool 705 may be permanently coupled to one another. That is, the surgical tool 705 and handle 704 may be integrally formed as a single component with one another and may not be decoupled. However, in other examples, the handle 704 and surgical tool 705 may be removably coupled to one another. The surgical tool may comprise a probe, biopsy needle, pin, scope, jamshidi, drill, drill guide, screwdriver, etc.

The handle 704 may include six walls: front wall 706, back wall 708, top wall 710, bottom wall 712, first side wall 514, and second side wall 716. The walls 706, 708, 710, 712, 714, and 716 may be curved as depicted in FIG. 7. However, in other examples, the walls of the handle 704 may be substantially flat and/or planar. The size, dimensions, and shape of the handle 704 may depend on the type of medical instrument 702. The front wall 706 may be positioned opposite the back wall 708 which is not visible in FIG. 7, defining a width of the handle 704. The top wall 710 may be positioned opposite the bottom wall 712, defining a height of the handle 704. Further, the first side wall 714 or first end 714 may be positioned opposite the second side wall 716 or second end 716, defining a length of the handle 704.

The surgical tool 705 is coupled to, and extends from, the first end 714 of the handle 704 to a tip 707 of the medical instrument 702. Thus, the tip 707 may define the end or physical extent of the surgical tool 705 and medical instrument 702.

The handle 704 may include a groove 732 for receiving one or more cables (e.g., cables 226 described above in FIGS. 2A and 2B) that electrically couple the tracking sensor 502 (not shown in FIG. 7) to a computing device (e.g., computing device 214 described in FIGS. 2A and 2B) of an image-guided surgery system (e.g., image-guided surgery system 100 described in FIG. 1).

The handle 704 comprises a tracking sensor interface 718. Tracking sensor interface 718 may also be referred to as tracking sensor platform 718 in the description herein. As depicted in the example of FIG. 7, the tracking sensor interface 718 may be integrally formed within the handle 704 as a recess. As such, tracking sensor interface 718 may also be referred to in the description herein as recess 718. However, other shapes, dimensions, and orientations may be used for the tracking sensor interface 718 such as grooves, openings, etc.

The tracking sensor interface 718 is configured to receive the tracking sensor 502. Thus, the tracking sensor interface 718 may be the portion of the handle 704 that interacts with and/or engages the tracking sensor 502. Specifically, the tracking sensor interface 718 comprises a first mating surface 720 that interfaces with and/or physically contacts the second mating surface 509 (not shown in FIG. 7) of the tracking sensor 502 to couple and decouple the tracking sensor 502 and medical instrument 702.

The first mating surface 720 comprises a first side wall 724, second side wall 722, bottom side wall which is formed by an interior surface of a portion of the bottom wall 712 of the handle 704, front wall which is formed by an interior surface of a portion of the front wall 706 of the handle 704, and a back wall which is formed by an interior surface of a portion of the back wall 708 of the handle 704. Thus, the tracking sensor interface 718 may extend between the front wall 706 and back wall 708 as depicted in FIG. 7. However, in other examples, the tracking sensor interface 718 may not extend between the walls 706 and 708.

Further, the handle 704 may include one or more magnets 734. Specifically, the handle 704 may include exactly two magnets. However, more or less than two magnets may be used. Further, one or more of the orientation, position, size, and strength of the magnets may be adjusted to adjust a magnetic field pattern produced by the magnets 734. The magnets 734 may be positioned in the tracking sensor interface 718 facing the magnetic field detectors 522 (not shown in FIG. 7) of the tracking sensor 502 (not shown in FIG. 7). For example, the magnets 734 may be included on the bottom wall 712 of the handle 704, proximate to and/or within the tracking sensor interface 718.

In the example of FIG. 7, an attachment device 726 may be physically and permanently coupled to the handle 704. Specifically the attachment device 726 may be physically and permanently coupled to the tracking sensor interface 718. In the example shown in FIG. 7, the attachment device 726 may comprise a pivotable flange that may be physically coupled to the second side wall 722 of the first mating surface 720. As such, attachment device 726 may also be referred to in the description herein as pivotable flange 726. The attachment device 726 may be adjusted to couple and decouple the tracking sensor 502 and medical instrument 702. For example, an external torqueing force, which may be provided by the operator, may pivot the flange 726 to selectively couple and decouple the tracking sensor 502 and medical instrument 702. As described in greater detail below with reference to FIG. 8, the attachment device 726 may engage the lip 520 (not shown in FIG. 7) of the tracking sensor 502 to couple the tracking sensor 502 and medical instrument 702, and may disengage the lip 520 to decouple the tracking sensor 502 and medical instrument 702. Specifically, the attachment device 726 may be adjusted between a first engaged or extended position where the tracking sensor 502 and medical instrument 702 are physically coupled to one another, and a second disengaged or compressed position where the tracking sensor 502 and medical instrument 702 are decoupled from one another.

The attachment device 726 may be naturally biased towards the extended position. Thus, when compressed, the attachment device 726 may exert an outward force, and when the compressive force ceases, the attachment device 726 may extend from the more compressed position to a more extended position without the application of any external forces. Thus, attachments device 726 may act in a similar manner to a spring.

Further, the first side wall 724 may include a ledge 730, which protrudes away from the surface of the first side wall 724 towards the second side wall 726. As described in greater detail below with reference to FIG. 8, the ledge 730 may engage the heel 518 (not shown in FIG. 7) of the tracking sensor 502 to stabilize the tracking sensor 502 when coupling the tracking sensor 502 to the medical instrument 702. Cutting plane 725 defines a cross-section of the medical instrument 702 shown in FIG. 8.

Turning now to FIG. 8, it shows a cross-sectional view 800 of an embodiment of a medical instrument assembly 801 where the attachment device 726 is included in the tracking sensor interface 718 of the handle 704 of the medical instrument 702. Medical instrument assembly 801 may be the same or similar to medical instrument assembly 208 described above with reference to FIGS. 2A and 2B. As such, medical instrument assembly 801 may be included in the electromagnetic surgical navigation system (e.g., electromagnetic tracking system 200 shown in FIGS. 2A and 2B). Medical instrument assembly 801 includes the tracking sensor 502 and the medical instrument 702. Specifically, FIG. 8 depicts the medical instrument 702 and tracking sensor 502 in the first engaged position, where the tracking sensor 502 and medical instrument 702 are coupled to one another.

In the engaged first position, the attachment device 726 of the handle 704 physically couples the tracking sensor 502 and the medical instrument 702, via interaction with the lip 520 of the tracking sensor 502. The attachment device 726 may pivot about a pivot point 804, where the attachment device 726 is physically coupled to the handle 704. Thus, the attachment device 726 may extend outward from the pivot point 804 to an end 806 of the device 726. As such, the end 806 may translate towards the first side wall 724 and away from the second side wall 722 to engage and couple the tracking sensor 502 and medical instrument 702. Further, the end 806 may translate away from the first side wall 724 and towards the second side wall 722 to disengage and decouple the tracking sensor 502 and medical instrument 702. Thus, by pivoting the attachment device 726, the tracking sensor 502 may be selectively coupled with and decoupled from the medical instrument 702.

The end 806 of the attachment device 726 may extend through the bottom wall 712 of the handle 704, and may protrude from the bottom of the handle 704. Thus an operator may pivot the attachment device 726, and therefore couple and decouple the tracking sensor 502 and medical instrument 702 by moving the end 806 of the attachment device 726.

The attachment device 726 may include a stop surface 808 that physically contacts the lip 520 of the tracking sensor 502. When the stop surface 808 and lip 520 are in physical contact as shown in the example of FIG. 8, the stop surface 808 may prevent and/or restrict movement of the stop surface 808 relative to the lip 520, thereby coupling the tracking sensor 502 to the medical instrument 702. Thus, by moving the attachment device 726, via the end 806 of the device 726, the stop surface 808 may be translated into contact with the lip 520 of the tracking sensor 502 to physically couple the tracking sensor 502 and medical instrument 702, and may be translated away from and out of contact with the lip 520, to decouple the tracking sensor 502 and medical instrument 702. Thus, the first engaged position may represent a position of the attachment device 726 where the stop surface 808 is physically contacting the lip 520 of the tracking sensor 502.

Further, as depicted in the example of FIG. 8, the ledge 730 of the first side wall 724 may be positioned over, and may physically contact the heel 518 of the tracking sensor 502, restricting movement of the tracking sensor 502 relative to the handle 704. When coupling the tracking sensor 502 to the medical instrument 702, the heel 518 may first be inserted below the ledge 730. The interaction of the heel 518 and ledge 730 may help to stabilize the tracking sensor 502 when coupling it to the medical instrument 702.

Once the heel 518 is inserted below the ledge 730, the lip 520 may be rotated down into contact with the flange 726. Specifically, the tracking sensor 502 may rotate via engagement of the heel 518 and ledge 730. Thus, the geometry of the heel 518 and ledge 730 may permit the tracking sensor 502 to pivot relative to the handle 704, when the heel 518 is inserted below the ledge 730.

As the lip 520 pivots and engages the flange 726, it may compress the flange 726 towards the second side wall 722 (e.g., to the right in FIG. 8). Then, once the lip 520 drops below the stop surface 808, the flange 726 may extend outwards towards first side wall 724, and the stop surface 808 may physically contact the lip 520 to couple the tracking sensor 502 and medical instrument 702.

A hand of an operator may hold the handle 704 at a handgrip 805. Thus, the handle 704 may include a handgrip 805 for receiving a hand of an operator. The handgrip 805 may be shaped to conform to the geometry of a hand. The operator may hold the handgrip 805 to manipulate and move the medical instrument 702. In some examples, the tracking sensor interface 718 may be included in the handgrip 805 of the handle 704. However, in other examples, the tracking sensor interface 718 may be included in the handle 704 at a location exterior to the handgrip 805. In some examples, the handgrip 805 may comprise only a portion of the handle 704. Thus, in such examples, a user's hand my only interact with and/or cover a portion of the handle 704. However, in other examples, the handgrip 805 may comprise substantially all of the handle 704. Thus, in such examples, a user's hand may interact with and/or cover substantially all of the handle 704.

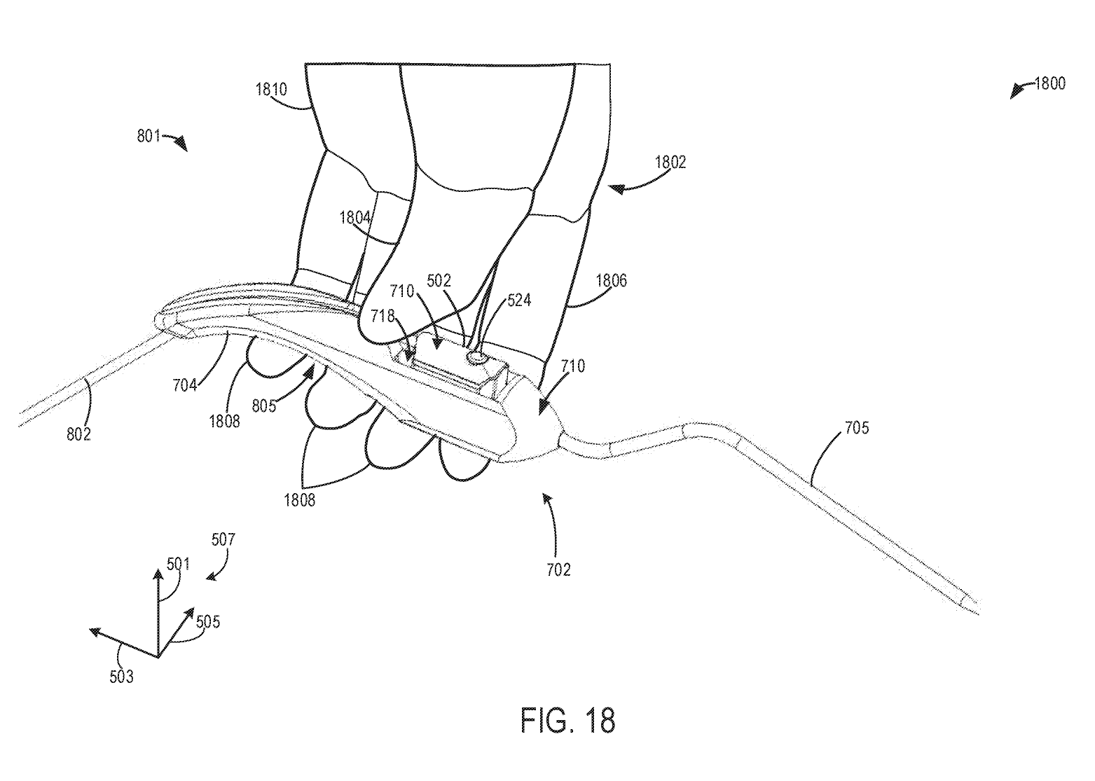

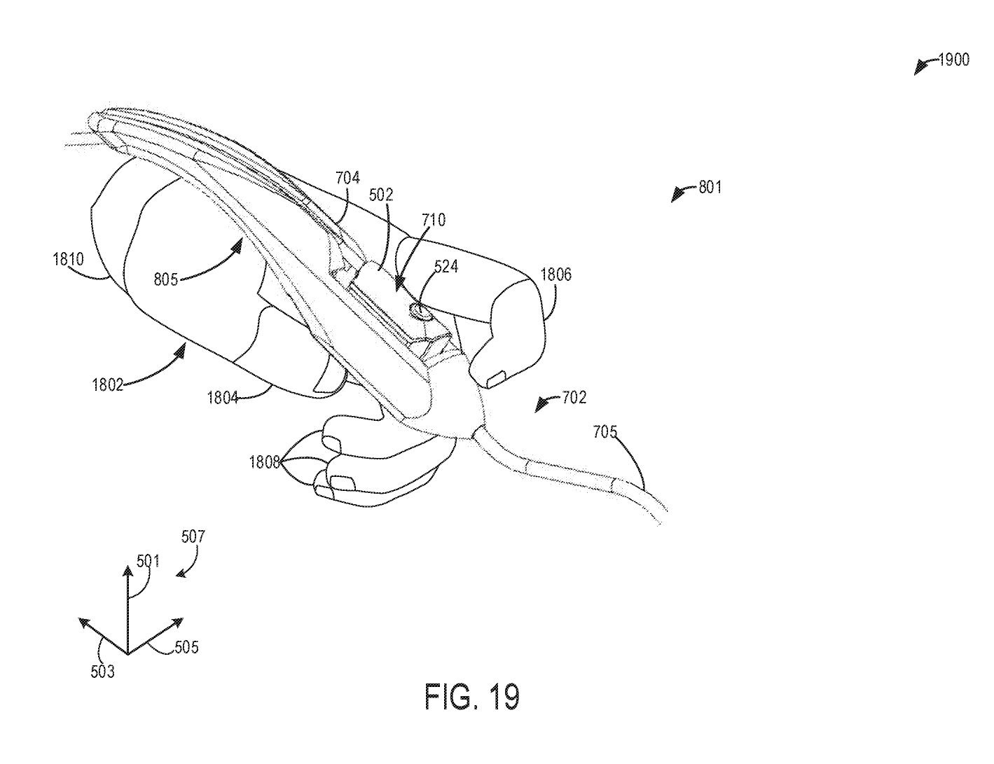

As depicted in the example of FIG. 8, the handgrip 805 may form a curved portion of the handle 704. That is, the handgrip 805 may be curved. An operator may hold the handgrip 805 such that the thumb of the operator is positioned over the user input device 524. In this way, the operator may manipulate the user input device 524 while holding and/or moving the medical instrument 702 without having to reposition their hand. A more detailed description of the positioning of a user's hand with respect to the handle 704 is described in greater detail below with reference to FIGS. 18-19. Thus, while performing surgery, the operator does not need to move or reposition their hand to manipulate the user input device 524. In this way, the user input device 524 may be positioned within the handle 704, such that it is accessible to the operator's fingers, in a natural holding position of the medical instrument 702.

For example, during surgery, the operator may press the user input device 524 to record movements of the medical instrument 702. Fingers of the operator may grip the handgrip 805 at the bottom wall 712 of the handle 704. Thus, the palm of the operator hand may be positioned over the handgrip 805, above top wall 710 of the handle 704. More simply, the operator's palm my sit on top of the handle 704, and their fingers may curl around the handgrip 805, on the bottom wall 712 of the handle 704. The thumb may remain above the handle 704, and may extend from the handgrip 805 over the top of the user input device 524.

Further, the operator's fingers may be positioned underneath the handle 704, proximate the end 806 of the attachment device 726. As such, the operator's fingers may provide torqueing force to the attachment device 726 for decoupling the tracking sensor 502 and medical instrument 702. In this way, the operator may pivot and/or manipulate the attachment device 726 to couple and/or decouple the tracking sensor 502 without having to reposition or move their hand. Specifically, to couple the tracking sensor 502 to the medical instrument, the operator may hold the handgrip 805 with one hand, while pushing (e.g., providing a downward force) the tracking sensor 502 into engagement with the tracking sensor interface 718 with their other hand. Specifically, the operator may push the lip 520 of the tracking sensor 502 past the stop surface 808 of the attachment device 726, until the attachment device 726 and lip 520 “click.” Thus, once the lip 520 drops below, and clears the stop surface 808 of the attachment device 726, the attachment device 726 may snap back towards the first side wall 510 of the tracking sensor 502, producing an auditory click, that may signal to the operator that the tracking sensor 502 is physically coupled to the medical instrument 702.

To decouple the tracking sensor 502 from the medical instrument 702, the operator may continue to hold the handgrip 805, and may only need to move one or more of their fingers along the bottom wall 712 of the handle 714 to engage the end 806 of the attachment device 726. The operator may pull the end 806 of the attachment device 726 towards 722 their palm and second side wall 722, to separate the attachment device 726 from the lip 520 of the tracking sensor 502. Once cleared from the stop surface 808 of the attachment device 726, the operator may then lift the tracking sensor 502 with their other hand, out from the tracking sensor interface 718, and out of engagement with the medical instrument 702.

Tracking sensor 502 may include electrical wire 802 which may be the same or similar to electrical cables 226 described above with reference to FIGS. 2A and 2B. The electrical wire 802 may extend out from the first side wall 510 of the housing 504 of the tracking sensor 502, via opening 610. The electrical wire 802 may electrically couple the electrical components of the tracking sensor 502, such as the circuit boards 606 and 608, with a computing device (e.g., computing device 214 described above in FIGS. 2A and 2B). The electrical wire 802 may be received in the groove 732 of the handle 704.

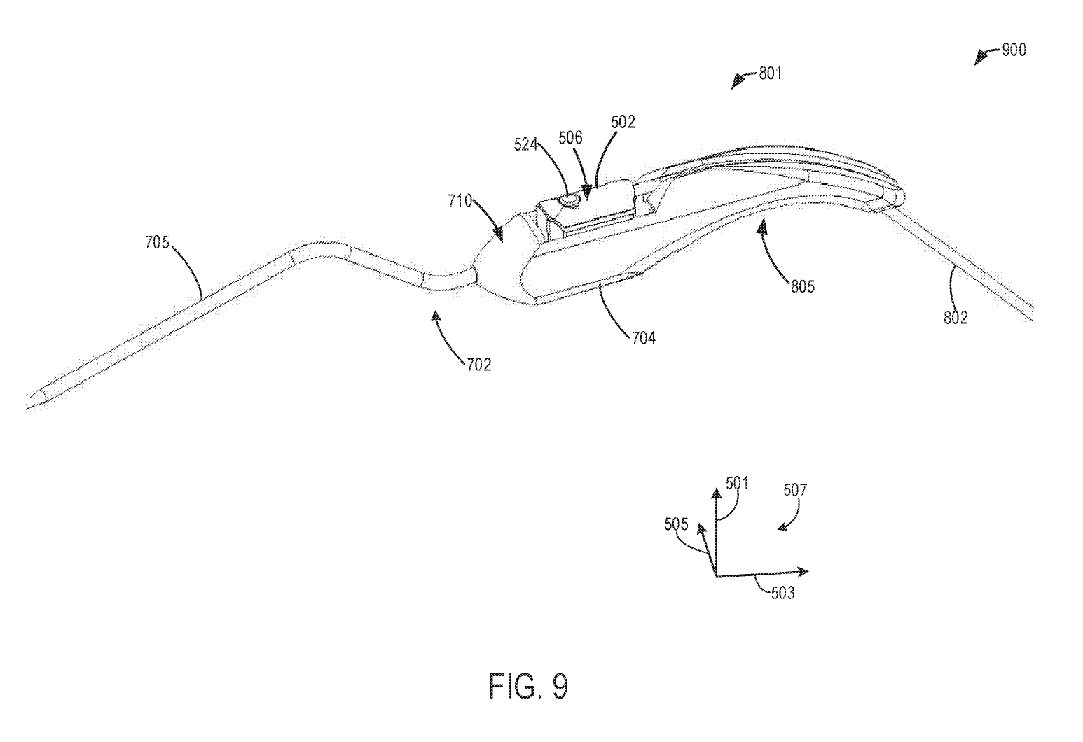

FIG. 9 shows an isometric view 900 of the first embodiment of the medical instrument assembly 801 described above in FIG. 8. Thus, FIG. 9 depicts the tracking sensor 502 and medical instrument 702 in the first engaged position, where the medical instrument 702 and tracking sensor 502 are coupled to one another. When coupled together, the top wall 506 of the tracking sensor 502 may extend above and/or may protrude from the top wall 710 of the handle 704. As such, the user input device 524 may be easily accessible and visible to an operator. That, is the user input device 524 may form a portion of an external surface of the medical instrument assembly 801, and as such, no additional components may separate the user input device 524 from exterior portions of the medical instrument assembly 801.

An operator's fingers may be positioned under the handgrip 805, with the palm above. Thus, the palm and fingers of the operator may extend around a circumference of the handgrip 805. The thumb of the operator's hand may extend towards the tool 705, over the user input device 524, for seamless manipulation thereof

Continuing to FIGS. 10-12, they depict a second example embodiment of the medical instrument assembly, where the attachment device is physically coupled to the tracking sensor. Beginning with FIG. 10, it shows an isometric view 1000 of an embodiment of the tracking sensor 502, where the attachment device 726 is physically and permanently coupled to the tracking sensor 502. Specifically, as shown in the example of FIG. 10, the attachment device 726 may be physically coupled to the first side wall 510 of the receiver housing 504.

The attachment device 726 is depicted in the example of FIG. 10, as an adjustable tab, that extends from the first side wall 510 of the receiver housing 504. Thus, the attachment device 726 may be included on the second mating surface 509. As such, attachment device 726 may also be referred to in the description herein as tab 726. The attachment device 726, may be physically coupled to the side wall 510 along one edge. Thus, three edges of the attachment device 726 may not be coupled to the side wall 510, permitting movement of the tab 726 relative to the tracking sensor 502. Specifically, the tab 726 may pivot about pivot axis 1004, where the pivot axis 1004 represents the point at which the tab 726 is physically coupled to the side wall 510.

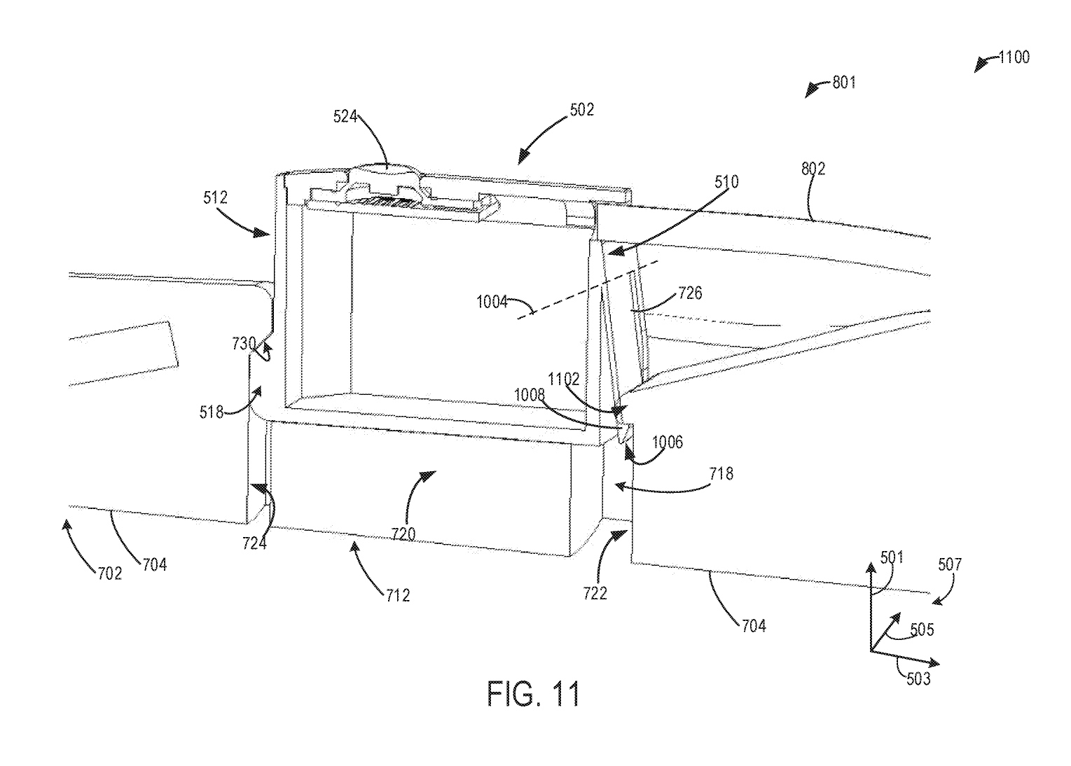

The tab 726 may extend away from the side wall 510 to an end 1006 of the tab 726. Thus, end 1006 may be the same or similar to end 806 described above with reference to FIG. 8. The end 1006 of the tab 726 may swing outwards, away from the side wall 510 about the pivot axis 1004. Further, the tab 726 may be compressed, by moving the end 1006 of the tab towards the side wall 510. As described in greater detail below with reference to FIG. 11, the tab 726 may initially be compressed by a mating component of the tracking sensor interface (e.g., lip 1102 described below in FIG. 11), and then may extend outwards to physically couple the tracking sensor 502 and medical instrument 702 (not shown in FIG. 10). More specifically, a stop surface 1008 of the tab 726, may engage a lip of the tracking sensor interface 718 to couple the tracking sensor 502 and medical instrument 702. Although the tab 726 is shown to be included on the side wall 510 in the example of FIG. 10, it should be appreciated that the tab 726 may be positioned on another wall of the housing 504, such as the second side wall 512.

Continuing to FIG. 11, it shows a cross-sectional view 1100 of the second embodiment of the medical instrument assembly 801, where the attachment device 726 is included on the tracking sensor 502. Similar, to the first embodiment of the medical instrument assembly 801 described above with reference to FIGS. 7-9, the heel 518 of the tracking sensor 502 may first be inserted below the ledge 730 of the handle 704. Then tracking sensor 502 may then pivot via the interfacing geometry of the heel 518 and ledge 730.

However, in the example of the medical instrument assembly 801 shown in FIG. 11, the attachment device 726 may be included on the tracking sensor 502. To couple the tracking sensor 502 and medical instrument 702, the attachment device 726 may initially be compressed, such that the end 1006 of the attachment device 726 is pushed towards the first side wall 510 of the tracking sensor interface 718.

As the tracking sensor 502 is pushed downwards towards the bottom wall 712 of the handle 704, the lip 1102 physically contacts the attachment device 726, and compresses the attachment device 726. Specifically lip 1102 may extend from the second side wall 722 of the tracking sensor interface 718 and compress the attachment device 726. Thus the end 1006 is pushed towards the side wall 510. Then, once the stop surface 1008 clears the lip 1102, the attachment device 726 may naturally pivot outwards, away from the side wall 510, towards the second side wall 722 of the tracking sensor interface 718, thereby physically coupling the tracking sensor 502 and medical instrument 702. Thus, the stop surface 1008 may be positioned below the lip 1002, and may physically contact the lip 1102 to hold the tracking sensor 502, and couple the tracking sensor 502 to the medical instrument 702.

In this way, when the stop surface 1008 of the attachment device 726 clears the lip 1102, the attachment device 726 may naturally extend outwards, and clip in the tracking sensor 502. In some examples, the attachment device 726 may produce an auditory “click” when it snaps into engagement with the lip 1102, thereby providing an indication to the operator when the tracking sensor 502 is physically coupled to the medical instrument 702.

To disengage the tracking sensor 502 and medical instrument 702, a compressive force may be applied to the attachment device 726, to push the attachment device 726 away from the lip 1002. Once cleared from the lip 1102, the tracking sensor 502 may be pulled upwards, away from, and out of engagement with the medical instrument 702. For example, the operator may push on the attachment device 726.

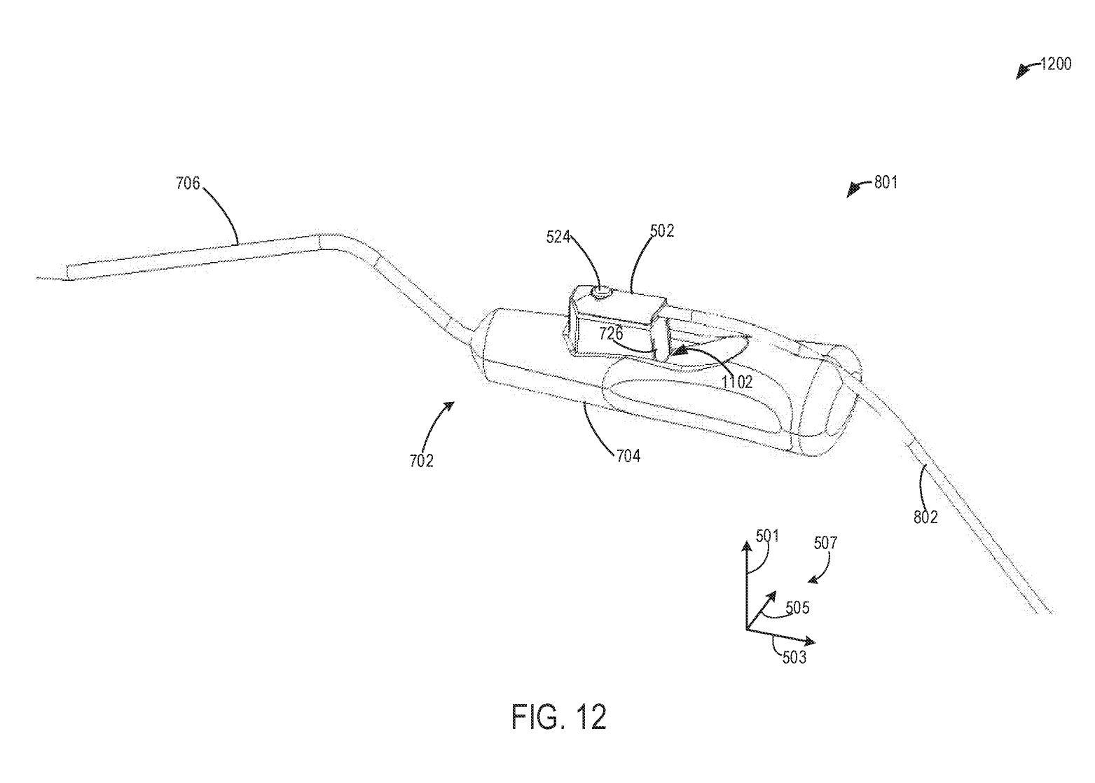

FIG. 12 shows an isometric view 1200 of the second embodiment of the medical instrument assembly 801 described above in FIG. 11, where the tracking sensor 502 is physically coupled to the medical instrument 702.

Turning now to FIGS. 13-15, they show examples of a third embodiment of the medical instrument assembly 801, where portions of the attachment device may be included on both the tracking sensor 502 and medical instrument 702. Focusing now on FIG. 13 it shows an embodiment of the tracking sensor 502, where the sensor 502 includes overhangs 1302, that may be formed on the corners of housing 504, where the bottom wall 508 and second side wall 512, meet with one of the front wall 514 and back wall 516. Thus, in the example shown in FIG. 13 two overhangs 1302 may be included, although only one is depicted in FIG. 13. However, it should be appreciated that the positioning of the overhangs 1302, and number of overhangs 1302 may be adjusted as desired.

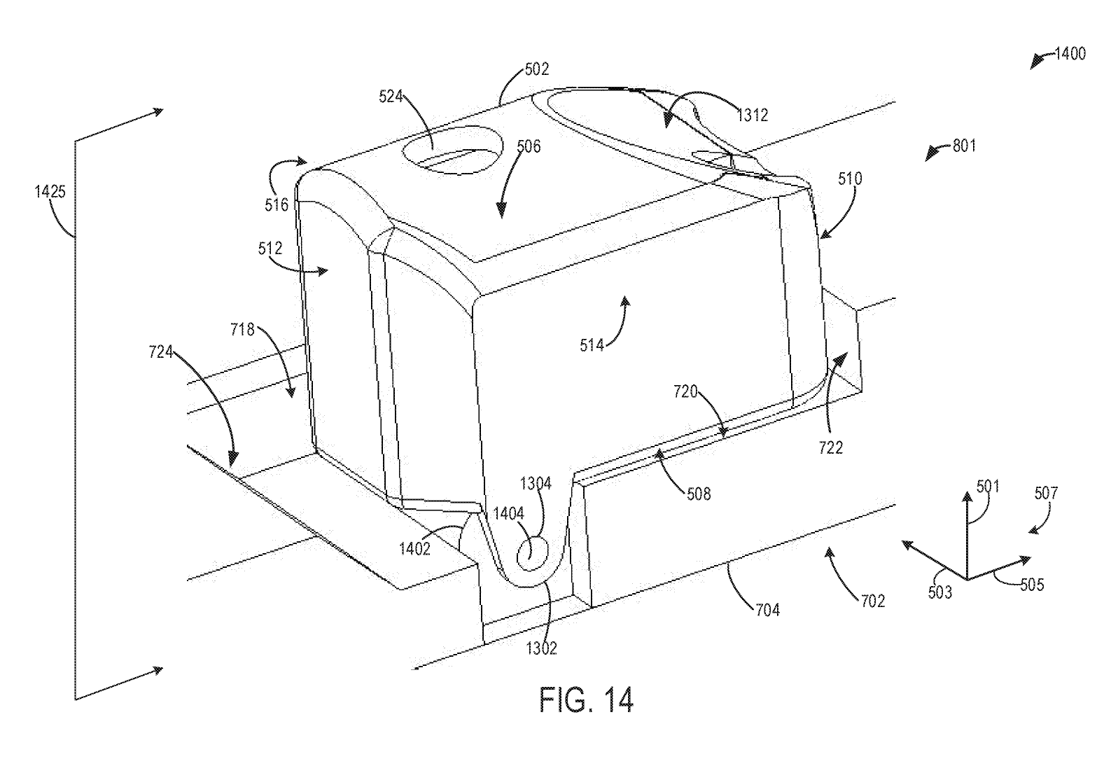

The overhangs 1302 may extend from the walls 514 and 516, below the bottom wall 508, along edges of the bottom wall 508. Thus, the overhangs 1302 may be flush with, and parallel to the front wall 514 and back wall 516. The overhangs 1302 may include apertures 1304 for receiving a pin of the medical instrument 702 (not shown in FIG. 13). Thus, the apertures 1304 may rotate relative to the pin (e.g., pin 1404 described below in FIG. 14), enabling rotation of the tracking sensor 502 about the pin.

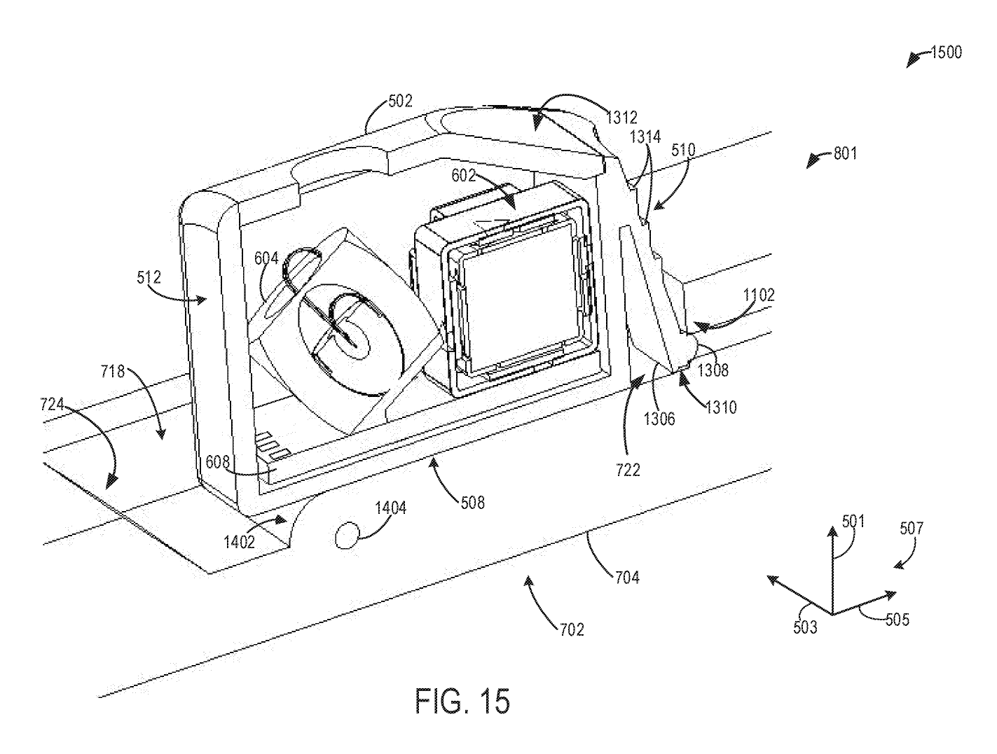

The tracking sensor 502 may further include a tab 1306 that may only be coupled to the side wall 510 along one edge. Thus, the other three edges of the tab 1306 may not be coupled to the tracking sensor 502, and the tab 1306 may pivot relative to the tracking sensor 502 about the edge that couples the tab 1306 to the tracking sensor 502. An end 1310 of the tab 1306 may extend away from the side wall 510, and may be displaced a greater distance than the rest of the tab 1306 when the tab 1306 is pivoted. The tab may include a knob 1308 that protrudes outwardly from the surface of the tab 1306. The knob 1308 may be positioned proximate to, or at the end 1310 of the tab 1306. The knob 1308 may engage a mating component of the handle 704 (not shown in FIG. 13), such as lip 1102 described below in FIG. 15, to physically couple the tracking sensor 502 and medical instrument 702.

Further, the tracking sensor 502 may include a finger recess 1312 along one or more of the top wall 506 and side wall 510. Thus, the finger recess 132 may be formed along a portion of the top wall 506 and side wall 510. The finger recess 1312 may be a curved surface that conforms to a shape of a thumb or finger. Further, the finger recess 1312 may include surface features 1314 that may be raised from the surface of the recess 1312 to aid an operator in positioning their finger in the recess 1312. In other examples, the surface features may be grooves.

When the tracking sensor 502 is physically coupled to the medical instrument 702, an operator's thumb may sit in the recess 1312, and may extend over the user input device 524. Thus, the recess 1312, may support the operator's thumb and provide support for it during surgery. In further examples, the recess 1312 may receive an operator's finger when coupling and decoupling the tracking sensor 502 from the medical instrument 702. The end of the operator's finger may extend away from the user input device 524, towards the end 1310 of the tab 1306 as explained in greater detail below with reference to FIG. 15.

Continuing to FIG. 14 it shows an isometric view 1400 of the third embodiment of the medical instrument assembly 801, where the tracking sensor 502 includes the overhangs 1302. In such examples, the tracking sensor interface 718 may include a pin 1404 that engages with the apertures 1304 of the overhangs 1302. Specifically, when coupling the tracking sensor 502 to the medical instrument 702, the operator may provide a downward force that pushes the overhangs 1302 over the pin 1404. The pin 1404, may provide an outward force that pushes the overhangs 1302 away from the walls 514 and 516. Once the apertures 1304 clear the pin 1404, and the pin 1404 fits within the apertures 1304, the overhangs 1302 may snap back towards the walls 514 and 516. Thus, the pin 1404 may extend through the apertures 1304 when the tracking sensor 502 and the medical instrument 702 are coupled to one another.

The pin 1404 may enable rotation of the tracking sensor 502. That is, the apertures 1304 may rotate relative to the pin 1404, such that the tracking sensor 502 may rotate about the axis of the pin 1404. Cutting plane 1425 defines a cross-section of the medical instrument assembly 801 show below in FIG. 15.

Moving on to FIG. 15, it shows a cross-sectional view 1500 of the third embodiment of the medical instrument assembly 801 described above with reference to FIG. 14, exposing internal components of the medical instrument assembly 801. Specifically cross-sectional view 1500 may be taken along cutting plane 1425 shown in FIG. 14. Thus, the overhangs 1302 of the tracking sensor 502 are not shown in FIG. 15, since the overhangs 1302 extend from the walls 514 and 516, and may not extend between the walls 514 and 516 from the bottom wall 508.

The tracking sensor 502 and medical instrument 702 are shown coupled to one another in the example of FIG. 15. Specifically, the knob 1308 of the tab 1306, is positioned below the lip 1102 of the handle 704. The knob 1308 and lip 1102 are therefore in physical contact with one another, restricting movement of the tracking sensor 502 and the medical instrument 702.

An operator may position their finger in the recess 1312, such that the end of their finger extends towards the knob 1308. Thus, their palm may be positioned more proximate the side wall 724. To couple the tracking sensor 502 and medical instrument 702, the operator may first push overhangs 1302 (not shown in FIG. 15) past the pin 1404, until the pin 1404 fits within the apertures 1304 (not shown in FIG. 15). Then, the operator may rotate the tracking sensor 502 about the pin 1404, and push the knob 1308 downwards, until it drops below and clears the lip 1102. The tab 1306 may then extend outward, engage the lip 1102, and clip in the tracking sensor 502, physically coupling the tracking sensor 502 and medical instrument 702.

To decouple the tracking sensor 502 and the medical instrument 702, the operator may again position their finger so that it extends to the knob 1308. However, the operator may pull the knob 1308 towards the side wall 510 of the tracking sensor with the end of their finger, away from and out of engagement with the lip 1102. Then the user may pull the tracking sensor 502 upwards and out of engagement with the tracking sensor interface 718 to completely decouple the tracking sensor 702 and medical instrument 702.

Turning now to FIGS. 16 and 17, they show a fourth embodiment of the medical instrument assembly 801, where the tracking sensor 502 includes the heel 518 described above with reference to FIGS. 5-12, and the tab 1306 described above with reference to FIGS. 13-15.

Focusing on FIG. 16 it shows an isometric view 1600 of an embodiment of the tracking sensor 502, where the tracking sensor includes the heel 518, such as the examples described above with reference to FIGS. 5-12. Thus, the tracking sensor 502 may be identical to the embodiment of the tracking sensor 502 described above with reference to FIGS. 13-15, except that instead of including the overhangs 1302, the embodiment of the tracking sensor 502 shown in FIG. 16 may include the heel 518. Cutting plane 1625 defines a cross-section of the tracking sensor 502 shown below in FIG. 17.

Continuing to FIG. 17, it shows the fourth embodiment of the medical instrument assembly 801, where the tracking sensor 502 includes the heel 518 and tab 1306. The process for coupling and decoupling the tracking sensor 502 and medical instrument 702 may be the same as in the examples described above in FIGS. 13-15, except that the heel 518 may first be pushed into engagement with the ledge 730, instead of first pushing the overhangs 1302 over the pin 1404. Thus, in FIG. 17, the tracking sensor 502 may rotate via the geometry of the mating surfaces of the ledge 730 and heel 518.

Further, FIG. 17 shows an example where the magnetic field detectors 522 are included along a bottom of the circuit board 608, between the circuit board 608 and an internal surface of the bottom wall 508 of the tracking sensor 502. Although only one row comprising three of the detectors 522 is shown in FIGS. 17 it should be appreciated that two rows each comprising three of the detectors 522 may be included, such that the tracking sensor 502 includes six of the detectors 522.

Turning now to FIGS. 18 and 19, they show example embodiments of how an operator (e.g., operator 106 described above in FIG. 1) may grip and hold the handle 704 of the medical instrument assembly 801.

FIG. 18, shows a schematic 1800 of a first example gripping position of the medical instrument 702 of the medical instrument assembly 801. Thus, an operator's hand 1802 may hold the medical instrument 702 in the example gripping position shown in FIG. 18. As shown in FIG. 18, an operator's thumb 1804 may be positioned above the top wall 710 of the medical instrument 702, proximate the tracking sensor 502 and input device 524. More specifically, the thumb 1804 may be positioned above the top wall 510 of the tracking sensor 502. In this way, the operator may manipulate the input device 524 during surgery via their thumb 1804 without having to reposition their hand on the handle 704 of the medical instrument 702.

An index finger 1806 of the operator's hand 1802, and one or more secondary fingers 1808 (e.g., ring finger, middle finger, and pinkie finger) may extend underneath the medical instrument 702, while a palm 1810 of the hand 1802 may remain above the medical instrument 702. Specifically, as shown in the example of FIG. 18, the secondary fingers 1808 may extend below the handgrip 805. In examples where the tracking sensor interface 718 is included in the portion of the handle 704 comprising the handgrip 805, the secondary fingers 1808 and index finger 1806 may extend below the handgrip 805 and tracking sensor interface 718. However in other examples, where the tracking sensor interface 718 is included in a portion of the handle 704 exterior to the handgrip 805, the fingers 1808 and index finger 1806 may extend below the handgrip 805 only, and not below the tracking sensor interface 718. As shown in the examples of FIG. 18, the handgrip 805 may comprise substantially all of the handle 704, and thus, the fingers 1806 and 1808 may extend below the handle 704, along a length of the handle. Thus, the fingers 1806 and 1808 may wrap around the handgrip 805, and the thumb 1804 may remain on top of the handgrip 805, positioned over the tracking sensor 502.

Turning now to FIG. 19, it shows a schematic 1900 of a second example gripping position of the medical instrument 702 of the medical instrument assembly 801. Thus, an operator's hand 1802 may hold the medical instrument 702 in the example gripping position shows in FIG. 19. The second example gripping position may also be referred to herein as a pencil grip position.

In the example gripping position shown in FIG. 19, the thumb 1804, and secondary fingers 1808 may be positioned below the handle 704 of the medical instrument 702. The index finger 1806 may be positioned above the handle 704, proximate the top wall 710 and input device 524 of the tracking sensor 502. In this way, the operator may manipulate the input device 524 during surgery via their index finger 1806 without having to reposition their hand on the handle 704 of the medical instrument 702. A portion of the handgrip 805 may rest on the base of the thumb 1804, and/or the palm 1810 of the hand 1802.

It should be appreciated that the hand 1802 may grip and/or extend around the handgrip 805 of the handle 704 to hold the medical instrument 702. In some examples, the handgrip 805 may comprise substantially all of the handle 704, and thus the hand 1802 may extend around substantially all of the handle 704 in a gripping position. Further, in such examples, the tracking sensor interface 718 may be included within the portion of the handle 704 comprising the handgrip 805. However, in other examples, the handgrip 805 may comprise only a portion of the handle 704. In such examples, the tracking sensor interface 718 may be included within a portion of the handle 704 exterior to the handgrip 805. Thus, the handgrip 805 may not include the tracking sensor interface.

Although the palm 1810, and fingers 1808 may remain positioned around the handgrip 804, one of the thumb 1804 and/or index finger 1806 may extend to a portion of the handle 704 not including the handgrip 805. Thus the thumb 804 and/or index finger 1806 may extend away from the handgrip 805, towards the tracking sensor 502. In this way, a user may manipulate the input device 524, even in examples where the handgrip 805 is not included in a portion of the handle 704 where the tracking sensor 718 interface is included.

Although mechanical attachment devices are disclosed in FIGS. 5-19 for coupling and decoupling the tracking sensor and medical instrument, it should be appreciated that other types of coupling arrangements may be used. For example, magnets may be included in the tracking sensor and medical instrument, and the tracking sensor and medical instrument may be physically coupled to one another via the magnetic attraction of the permanent magnets included in the tracking sensor and medical instrument.

A technical effect of the disclosure is reducing surgery operating durations by automatically detecting a type of medical instrument via a magnetic field pattern generated by the medical instrument, and calibrating a tracking sensor based on the detected magnetic field pattern. Identifying the type of medical instrument via the magnetic field detection, reduces time an operator may spend searching for and verifying the medical instrument type. Another technical effect of the disclosure is reducing the cost of surgical equipment. By allowing for the tracking sensor to be coupled and decoupled to various types of medical instruments via a single interface, the tracking sensor design may be the same or similar for use in any medical instrument. Thus, the medical instruments may include a common tracking sensor interface that may receive a single type of sensor design, thus reducing the number of sensor designs and complexity of such systems. Further, by including a user interface button on the tracking sensor, a surgeon or other operator may manipulate the tracking sensor during surgery without having to reposition or adjust their hand on the medical instrument.

A medical instrument assembly for an electromagnetic surgical navigation system may comprise a tracking sensor interface included within a handle of a medical instrument, the interface having a first mating surface, a tracking sensor including one or more electromagnetic coils, and a second mating surface, and an attachment device, adjustable to physically couple and decouple the tracking sensor and medical instrument via the first and second mating surfaces. In the above medical instrument assembly the handle may include two or more magnets, where the magnets may produce a magnetic field signature at the interface, and where the magnetic field signature may be unique to each type of medical instrument. In any one or more of the above embodiments of the medical instrument assembly, the tracking sensor may further comprise a plurality of Hall effect sensors for detecting a magnetic field signature of the medical instrument. In any one or more of the above embodiments of the medical instrument assembly, the tracking sensor may further include one or more user interface buttons for one or more of: verifying physical coupling of the tracking sensor and medical instrument, verifying a type of the medical instrument, powering on the tracking sensor, toggling through menu options on a display screen, selecting an activity mode, calibrating the tracking sensor, and saving instrument trajectory routes and anatomical features. In any one or more of the above embodiments of the medical instrument assembly the user interface buttons may include lights for indicating one or more of an operating mode of the tracking sensor, a power state of the tracking sensor, and physical coupling of the tracking sensor and the medical instrument. In any one or more of the above embodiments of the medical instrument assembly, the attachment device may comprise a pivotable flange that may be adjustable between a compressed second position and an extended first position, where in the first position, the tracking sensor and medical instrument may be physically coupled to one another, and where in the second position the tracking sensor and medical instrument may be decoupled from one another. In any one or more of the above embodiments of the medical instrument assembly the attachment device may be permanently physically coupled to the tracking sensor. In any one or more of the above embodiments of the medical instrument assembly, the attachment device may be permanently physically coupled to the handle of the medical instrument. In any one or more of the above embodiments of the medical instrument assembly the attachment device may comprise a first portion permanently physically coupled to the tracking sensor and a second portion permanently physically coupled to the medical instrument. In any one or more of the above embodiments of the medical instrument assembly the tracking sensor may be configured as an electromagnetic receiver that detects electromagnetic waves generated by an electromagnetic transmitter.

In another representation, an electromagnetic medical navigation system may comprise a surgical instrument comprising a handgrip, the handgrip including an array of magnets and a coil assembly platform, where the array of magnets generates a magnetic field at the coil assembly platform, a first electromagnetic coil assembly removably coupled to the coil assembly platform for estimating a position of the surgical instrument within a patient, the coil assembly including a plurality of hall effect sensors for detecting the magnetic field, a controller in electrical communication with the coil assembly, the controller including computer-readable instructions stored in non-transitory memory for determining one or more of a manufacturer, type, and size of the surgical instrument based on the magnetic field, and a display screen for displaying the position of the surgical instrument within the patient. In some examples, the electromagnetic coil assembly of the above medical navigation system may further include a user interface button for receiving user input and providing feedback to a user based on a current operating state of the surgical instrument. Any one or combination of the above medical navigation systems, may further comprising, a second electromagnetic coil assembly coupled to anatomy of the patient. In any one or combination of the above medical navigation systems the first electromagnetic coil assembly may include one or more first electromagnetic coils and may be configured as an electromagnetic receiver and the second electromagnetic coil assembly may include one or more second electromagnetic coils and may be configured as an electromagnetic transmitter, and where a position of the surgical instrument is estimated based on outputs from one or more of the first electromagnetic coil assembly and the second electromagnetic coil assembly, where the first electromagnetic coils are adapted to sense an electromagnetic field produced by the second electromagnetic coil, and the second electromagnetic coils are adapted to sense an electromagnetic field produced by the first electromagnetic coils. In any one or combination of the above medical navigation systems, the first electromagnetic coil assembly may include one or more first electromagnetic coils and may be configured as an electromagnetic transmitter and the second electromagnetic coil assembly may include one or more second electromagnetic coils and may be configured as an electromagnetic receiver, and where a position of the surgical instrument is estimated based on outputs from one or more of the first electromagnetic coil assembly and the second electromagnetic coil assembly, where the first electromagnetic coils are adapted to sense an electromagnetic field produced by the second electromagnetic coil, and the second electromagnetic coils are adapted to sense an electromagnetic field produced by the first electromagnetic coils.

In yet further representations, a method may comprise detecting a magnetic field signature of an array of magnets included within a medical tool via an electromagnetic coil assembly coupled to the medical tool, identifying the medical tool type based on the magnetic field signature, calibrating outputs from the electromagnetic coil assembly based on the medical tool type, and correcting estimations of a position of a tip of the medical tool based on the calibrated outputs. In some examples of the above method, the magnetic field signature is unique to the type of medical tool. Any one or more of the above methods may further comprise receiving wireless signals from an electromagnetic transmitter and determining one or more of a position and orientation of the electromagnetic coil assembly based on the received wireless signals. Any one or more of the above methods may further comprise, notifying a user when the electromagnetic coil assembly is physically coupled to the medical tool via one or more of lights, vibration, and sounds. In any one or more of the above methods, the calibrating outputs from the electromagnetic coil assembly may further be based on product information of the medical tool to which the coil assembly is removably coupled, where the product information may include one or more of the size, length, dimensions, specifications, weight, manufacturer, product identification number, manufacturing date, supplier contact information, and serial number of the medical tool.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A medical instrument assembly for an electromagnetic surgical navigation system, comprising:
a tracking sensor interface included within a handle of a medical instrument, the interface having a first mating surface;
a tracking sensor including one or more electromagnetic coils, and a second mating surface; and
an attachment device, adjustable to physically couple and decouple the tracking sensor and medical instrument via the first and second mating surfaces.

2. The assembly of claim 1, wherein the handle includes two or more magnets, and where the magnets produce a magnetic field signature at the interface, the magnetic field signature unique to each type of medical instrument.

3. The assembly of claim 1, wherein the tracking sensor further comprises a plurality of Hall effect sensors for detecting a magnetic field signature of the medical instrument.

4. The assembly of claim 1, wherein the tracking sensor further includes one or more user interface buttons for one or more of: verifying physical coupling of the tracking sensor and medical instrument, verifying a type of the medical instrument, powering on the tracking sensor, toggling through menu options on a display screen, selecting an activity mode, calibrating the tracking sensor, and saving instrument trajectory routes and anatomical features.

5. The assembly of claim 4, wherein the user interface buttons include lights for indicating one or more of an operating mode of the tracking sensor, a power state of the tracking sensor, and physical coupling of the tracking sensor and the medical instrument.

6. The assembly of claim 1, wherein the attachment device comprises a pivotable flange that is adjustable between a compressed second position and an extended first position, where in the first position, the tracking sensor and medical instrument are physically coupled to one another, and where in the second position the tracking sensor and medical instrument are decoupled from one another.

7. The assembly of claim 1, wherein the attachment device is permanently physically coupled to the tracking sensor.

8. The assembly of claim 1, wherein the attachment device is permanently physically coupled to the handle of the medical instrument.

9. The assembly of claim 1, wherein the attachment device comprises a first portion permanently physically coupled to the tracking sensor and a second portion permanently physically coupled to the medical instrument.

10. The assembly of claim 1, wherein the tracking sensor is configured as an electromagnetic receiver that detects electromagnetic waves generated by an electromagnetic transmitter.

11. An electromagnetic medical navigation system comprising:
a surgical instrument comprising a handgrip, the handgrip including an array of magnets and a coil assembly platform, where the array of magnets generates a magnetic field at the coil assembly platform;
a first electromagnetic coil assembly removably coupled to the coil assembly platform for estimating a position of the surgical instrument within a patient, the coil assembly including a plurality of hall effect sensors for detecting the magnetic field;
a controller in electrical communication with the coil assembly, the controller including computer-readable instructions stored in non-transitory memory for determining one or more of a manufacturer, type, and size of the surgical instrument based on the magnetic field; and
a display screen for displaying the position of the surgical instrument within the patient.

12. The system of claim 11, wherein the electromagnetic coil assembly further includes a user interface button for receiving user input and providing feedback to a user based on a current operating state of the surgical instrument.

13. The system of claim 11 further comprising, a second electromagnetic coil assembly coupled to anatomy of the patient.

14. The system of claim 14, wherein the first electromagnetic coil assembly includes one or more first electromagnetic coils and is configured as an electromagnetic receiver and the second electromagnetic coil assembly includes one or more second electromagnetic coils and is configured as an electromagnetic transmitter, and where a position of the surgical instrument is estimated based on outputs from one or more of the first electromagnetic coil assembly and the second electromagnetic coil assembly, where the first electromagnetic coils are adapted to sense an electromagnetic field produced by the second electromagnetic coil, and the second electromagnetic coils are adapted to sense an electromagnetic field produced by the first electromagnetic coils.

15. The system of claim 14, wherein the first electromagnetic coil assembly includes one or more first electromagnetic coils and is configured as an electromagnetic transmitter and the second electromagnetic coil assembly includes one or more second electromagnetic coils and is configured as an electromagnetic receiver, and where a position of the surgical instrument is estimated based on outputs from one or more of the first electromagnetic coil assembly and the second electromagnetic coil assembly, where the first electromagnetic coils are adapted to sense an electromagnetic field produced by the second electromagnetic coil, and the second electromagnetic coils are adapted to sense an electromagnetic field produced by the first electromagnetic coils.

16. A method comprising:
detecting a magnetic field signature of an array of magnets included within a medical tool via an electromagnetic coil assembly coupled to the medical tool;
identifying the medical tool type based on the magnetic field signature;
calibrating outputs from the electromagnetic coil assembly based on the medical tool type; and
correcting estimations of a position of a tip of the medical tool based on the calibrated outputs.

17. The method of claim 15, wherein the magnetic field signature is unique to the type of medical tool.

18. The method of claim 15, further comprising receiving wireless signals from an electromagnetic transmitter and determining one or more of a position and orientation of the electromagnetic coil assembly based on the received wireless signals.

19. The method of claim 15 further comprising, notifying a user when the electromagnetic coil assembly is physically coupled to the medical tool via one or more of lights, vibration, and sounds.

20. The method of claim 15 wherein the calibrating outputs from the electromagnetic coil assembly is further based on product information of the medical tool to which the coil assembly is removably coupled, where the product information includes one or more of a size, length, dimensions, specifications, weight, manufacturer, product identification number, manufacturing date, supplier contact information, and serial number of the medical tool.
